(19) United States
(12) Patent Application Publication
(10) Pub. No.: US 2025/0099585 A1
(43) Pub. Date: Mar. 27, 2025

(54) IPSC-Based Gamma-Delta T-Cells, Compositions and Methods of Use Thereof

(71) Applicant: IN8bio, Inc., New York, NY (US)

(72) Inventors: Yanjie LI, Homewood, AL (US); Lei DING, Vestavia Hills, AL (US); Lawrence S. LAMB, Jr., Birmingham, AL (US)

(21) Appl. No.: 18/909,285

(22) Filed: Oct. 8, 2024

Related U.S. Application Data
(63) Continuation of application No. PCT/US23/18127, filed on Apr. 11, 2023.
(60) Provisional application No. 63/329,633, filed on Apr. 11, 2022.

(51) Int. Cl. A61K 39/00; C07K 14/435; C07K 14/725; C12N 5/0783; C12N 15/86

(52) U.S. Cl. CPC A61K 40/405; A61K 40/11; A61K 40/31; C07K 14/43522; C07K 14/7051; C12N 5/0636; C12N 15/86; C12N 2506/45; C12N 2740/15043

(57) ABSTRACT

Described are methods of preparing precursor cell-derived induced pluripotent stem cells (iPSCs) and methods of generating iPSC-derived gd T-cells. The invention also includes cell populations comprising the iPSCs, cell populations comprising the iPSC-derived gd T-cells, pharmaceutical compositions comprising the cell populations, and methods of treating cancer comprising administration of the cell population or pharmaceutical composition thereof.

### RELATED APPLICATIONS

This application is a continuation of International Application No. PCT/US23/18127, which designated the United States and was filed on Apr. 11, 2023, published in English, which claims the benefit of U.S. Provisional Application No. 63/329,633 filed Apr. 11, 2022. The entire contents of the above applications are incorporated herein by reference.

### BACKGROUND OF THE INVENTION

Gamma-delta (γδ) T-cells are naturally occurring immune cells that embody properties of both the innate and adaptive immune systems and represent ideal sources for developing allogeneic cell therapies for cancer, principally due to their ability to broadly recognize and kill tumor cells without the need for prior priming or the risk of initiating severe graft-versus-host disease (GvHD). In particular, γδ T-cells appear to have the ability to differentiate between healthy and tumor tissues, making them particularly attractive for adoptive cellular therapies against solid tumors. Early clinical trials have revealed that adoptively transferred γδ T-cells are well tolerated and, so far, appear to be safe when used as immunotherapies.

However, the challenge in utilizing these cells in therapeutic development is that the expansion of γδ T-cells can be difficult. As a minor lymphocyte population, starting cell counts are lower, expansion efficiency of v T-cells varies among donors, and, in some cases, ex vivo expansion and activation increases exhaustion and reduces the durability of γδ T-cells. Therefore, there remains a need in the art for an alternative source for γδ T-cells that can be banked and that overcomes the limitations of ex vivo expansion and activation of γδ T-cells.

### SUMMARY OF THE INVENTION

The present invention is based, at least partially, on the discovery that these limitations can be overcome by reprogramming precursor cells to induced pluripotent stem cells (iPSCs) which can then be differentiated to hematopoietic progenitor cells (HPCs), progenitor T-cells and cytotoxic T lymphocytes, including γδ cells. iPSCs possess the property of unlimited self-renewal and multi-lineage differentiation potential. Additionally, genetic modification or genome editing of iPSC-derived gd T-cells as well as selection and propagation of specific effector clones represents a source of near-limitless and rejuvenated immune cells. This disclosure encompasses methods of preparing precursor cell-derived induced pluripotent stem cells (iPSCs) (“precursor cell-derived iPSCs”), methods of generating iPSC-derived gd T-cells, a cell population comprising the precursor cell-derived iPSCs, a cell population comprising the HPCs differentiated from the precursor cell-derived iPSCs, a cell population comprising progenitor T-cells differentiated from the HPCs, a cell population comprising the iPSC-derived gd T-cells, pharmaceutical compositions comprising the cell populations described herein, and methods of treating cancer comprising administration of the cell population or pharmaceutical composition.

This disclosure encompasses a method of generating functional iPSC-derived gd T-cells comprising the steps of:
a. preparing a population of precursor cell-derived induced pluripotent stem cells (iPSCs), the method comprising the steps of:
   i. isolating a population of precursor cells and culturing the precursor cells in a culturing medium, wherein the precursor cells are human somatic cells;
   ii. transducing the precursor cells with a non-integrating virus vector encoding a plurality of reprogramming factors, the plurality optionally comprising one or more of Oct3/4, Sox2, Kfl4 and c-Myc; and
   iii. culturing the transduced precursor cells under conditions suitable for reprogramming the cells to pluripotency to obtain the population of precursor cell derived-iPSCs; and
b. differentiating the precursor cell-derived iPSCs to CD34+ hematopoietic progenitor cells (HPCs) and generating progenitor T-cells and functional iPSC-derived gd T-cells therefrom under feeder-free conditions, wherein the iPSC-derived gd T-cells comprise gd1 T-cells and/or gd2 T-cells.

Non-limiting examples of precursor cells are cord blood cells, bone marrow cells, skin cells, and lymphocytes. For example, the lymphocytes, such as gd T-cells and NK cells, can be isolated from cord blood, peripheral blood, or bone marrow. An additional example of a precursor cell is a CD34+ hematopoietic stem cells (HSCs) or CD34+ bone marrow cells. In certain additional aspects, the precursor cell is a primary skin cell (a primary keratinocyte).

Progenitor T-cells are generated from the HPCs. gd T-cells can then be generated from the progenitor T-cells by a multi-step differentiation process. In certain aspects, the multi-step differentiation process is conducted under feeder-free conditions and/or serum-free conditions.

The disclosure also includes a method of preparing a population of precursor cell-derived induced pluripotent stem cells (iPSCs) comprising:
i. isolating a population of precursor cells and culturing the precursor cells in a culturing medium, wherein the precursor cells are human somatic cells;
ii. transducing the precursor cells with a non-integrating virus vector encoding a plurality of reprogramming factors, optionally comprising one or more of Oct3/4, Sox2, Kfl4 and c-Myc; and
iii. culturing the transduced precursor cells under conditions suitable for reprogramming the cells to pluripotency to obtain the population of precursor cell derived-iPSCs.

The invention additionally encompasses a method of generating functional gd T-cells comprising: (i) generating progenitor T-cells from CD34+ hematopoietic progenitor cells (HPCs); and (ii) generating the gd T-cells from the progenitor T cells. In certain aspects, the differentiation steps are conducted under feeder-free conditions and/or serum-free conditions. In yet additional aspects, the functional gd T-cells comprise gd1 T-cells and/or gd2 T-cells.

The invention additionally encompasses a cell population comprising precursor cell derived-iPSCs, a cell population comprising the CD34+ hematopoietic progenitor cells (HPCs)(for example, iPSC-derived CD34+ HPCs), a cell population comprising the progenitor T-cells, and a cell population comprising functional iPSC-derived gd T-cells, wherein each of the cell populations is prepared by a method described herein. The invention further encompasses a cell population comprising precursor cell derived-iPSCs, a cell population comprising iPSC-derived CD34+ HPCs, a cell population comprising the progenitor T-cells, and a cell population comprising functional iPSC-derived gd T-cells. The invention also encompasses a population of precursor cell derived-iPSCs, a population of CD34+ hematopoietic progenitor cells (HPCs)(for example, iPSC-derived CD34+ HPCs), a population of progenitor T-cells and a population of functional iPSC-derived gd T-cells prepared by a method described herein. In yet further aspects, the precursor cell-derived iPSCs, such as gd T-iPSCs, are engineered to express a polypeptide selected from the group consisting of a chimeric antigen receptor (CAR), a survival factor, and a combination thereof. In certain aspects, the precursor cell-derived iPSCs comprise a transgene or polynucleotide that encodes a polypeptide selected from the group consisting of a chimeric antigen receptor (CAR), a survival factor, and a combination thereof. In additional aspects, the iPSC-derived gd T-cells express a polypeptide selected from the group consisting of a chimeric antigen receptor (CAR), a survival factor, and a combination thereof; or comprise or express a transgene or a polynucleotide that encodes the polypeptide. In certain aspects, the CAR is directed to a tumor antigen. In additional examples, the CAR comprises an extracellular antigen-binding domain comprising a chlorotoxin (CLTX) peptide; for example, comprising one or more CLTX peptides. In yet additional aspects, the polypeptide is a survival factor, for example, a polypeptide that confers resistance to a chemotherapeutic agent. Non-limiting examples of such polypeptides are alkyl guanine transferase (AGT), 06 methylguanine DNA methyltransferase (MGMT), P140K MGMT, L22Y-DHFR, thymidylate synthase, dihydrofolate reductase, multiple drug resistance-1 protein (MDR1), 5′ nucleotidase II, dihydrofolate reductase, and thymidylate synthase. In additional examples, the polypeptide confers resistance to a chemotherapeutic agent selected from the group consisting of trimethotrexate, temozolomide, raltitrexed, S-(4-Nitrobenzyl)-6-thioinosine, 6-benzyguanidine, nitrosoureas, fotemustine, cytarabine, and camptothecin.

Also included are pharmaceutical compositions comprising a cell population as described herein, for example, a cell population comprising functional gd T cells (e.g., iPSC-derived gd T cells), as well as methods of treating cancer or tumor in a patient in need thereof. The method can further comprise co-administering to said subject the chemotherapeutic agent in an amount sufficient to increase stress antigen expression on the cancer or tumor cells and wherein the cells express a polypeptide that confers resistance to the chemotherapeutic agent.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic of manufacturing strategies for iPSC-derived Vd1+ and Vd2+ gd T cells. Precursor cells from healthy donors were obtained and reprogrammed into gd T-iPSCs. After genotyping and characterization, both Vd1T- and Vd2T-iPSC clones were obtained, which were expanded and underwent a feed-free multi-step differentiation process to generate functional iPSC-gd T cell. Additional cell and disease specific gene-edits to the Vd1T- and Vd1T-iPSCs can be made.

FIG. 2 shows the results of multiplex genomic PCR assays and Sanger sequencing to examine rearrangements at TCRG and TCRD gene loci. The selected Vd1 T-iPSC clone was identified as Vg4- to Jg1/2 and Vd1-to-Jd1 recombination, and the selected Vd2 T-iPSC clones were Vg2- to Jg1/2 and Vd2- to Jd1.

FIG. 3 shows G-band karyotype analysis showing that all selected gd T-iPSC clones have normal karyotypes.

FIG. 4 shows flow cytometric analysis; the gd T-iPSC clones highly express the pluripotency markers SSE4 and OCT3/4.

FIGS. 5A-5C shows flow cytometric analysis after Vd1 T-iPSCs were expanded and differentiated with a feeder free culture system. At different stages, high yields of CD34+/CD34+ HPCs (86.9%) (FIG. 5A), CD5−/CD7+ progenitor T cells (75.4%) (FIG. 5B) and CD3+/gd TCR+ igd T cells (FIG. 5C) were obtained. All the iPSC differentiated Vd1 T cells have Vd1 TCRs (P<0.0001) (FIG. 5C).

FIG. 6A shows flow cytometric analysis of cells co-cultured with U87-GFP cells (glioblastoma cell line) at different ratios followed by staining with GFP and 7-AAD. GFP+/7-AAD+ cell percentages indicate cytotoxicity.

FIG. 6B shows a graph of percent cytotoxicity of i-Vd1T cells (iPSC-derived Vd1 T cells) against U87-GFP cells at different effector to target ratios (E:T). i-Vd1 T cells displayed high cytotoxicity against U87-GFP (glioblastoma cell line) in a dose-dependent manner.

FIG. 7 are images of U87-GFP cells co-cultured with i-Vd1 T cells at E:T=5:1 taken at different time points. Images were taken every minute (min) for a total of 300 min. Panel images taken at different time points shows U87-GFP cells were killed by i-Vd1 T cells during co-culture.

FIG. 8 is a schematic showing genome-modification and multi-step differentiation process. FIG. 8 also shows flow cytometric analysis showing that modified Vd1 T-iPSCs were transduced with 2×CLTX-noZ-MGMT CAR (divalent chlorotoxin chimeric antigen receptor with no signaling domain and MGMT) lentiviral vector with high efficiency (70%). These genome modified Vδ1 T-iPSC will be cloned and characterized before going through the multi-step differentiation process to further enhance the targeting and cytotoxicity of i-Vδ1 T cells against GBM and other solid tumors.

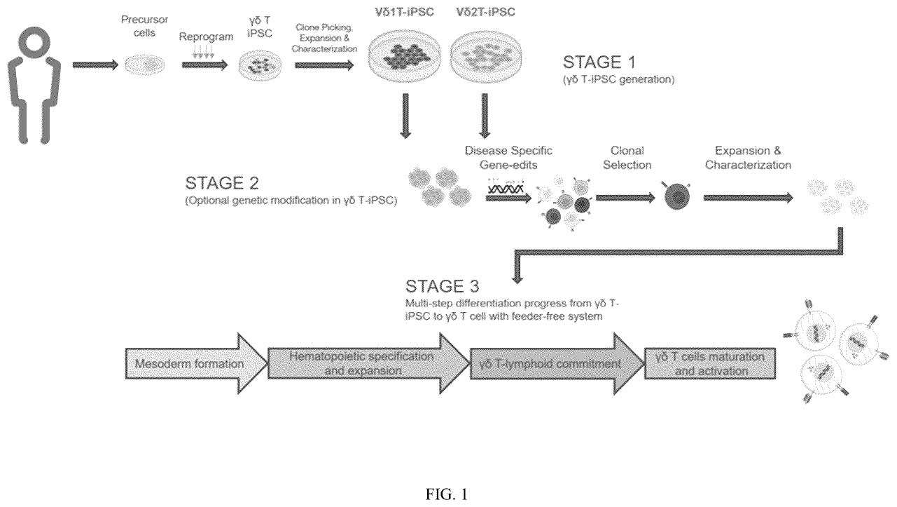

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

As used in the specification and the appended claims, the singular forms “a,” “an,” and “the” include plural referents unless the context clearly dictates otherwise. Thus, for example, reference to “a cell” includes a plurality of cells. In this specification and in the claims that follow, reference will be made to a number of terms that shall be defined to have the following meanings unless a contrary intention is apparent.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt. % to about 5 wt. %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”. Numbers, ratios, concentrations, amounts, ranges and other numerical data should be construed as modified by the term “about” unless inconsistent with the context.

As used herein, the term “engineered” with respect to a cell refers to a cell that has been genetically modified by the addition of exogenous genetic material in the form of DNA or RNA to the total genetic material of the cell. Such engineering encompasses the set of technologies used to change the genetic makeup of a cell. Genome editing, or genomic editing, or genetic editing, as used interchangeably herein, is a type of genetic engineering in which DNA is inserted, deleted, and/or replaced in the genome of a targeted cell. Targeted genome editing (interchangeable with “targeted genomic editing” or “targeted genetic editing”) enables insertion, deletion, and/or substitution at pre-selected sites in the genome. When an endogenous sequence is deleted or disrupted at the insertion site during targeted editing, an endogenous gene comprising the affected sequence can be knocked-out or knocked-down due to the sequence deletion or disruption. Therefore, targeted editing can also be used to disrupt endogenous gene expression with precision. Similarly used herein is the term “targeted integration,” referring to a process involving insertion of one or more exogenous sequences at pre-selected sites in the genome, with or without deletion of an endogenous sequence at the insertion site.

As used herein, a “transgene” refers to any nucleic acid sequence introduced into a cell, for example, by experimental manipulation, and which encodes a polypeptide of interest. The terms “transgene,” “polynucleotide” and “exogenous polynucleotide,” “exogenous gene,” and “heterologous gene,” and the like may be used interchangeably herein. The term “exogenous” is intended to mean that the referenced molecule or the referenced activity is introduced into the host cell, for example, by experimental manipulation.

The precursor cells can be mammalian somatic cells. Mammalian somatic cells can, for example, be human cells, non-human primate cells, or mouse cells. Non-limiting examples of somatic mammalian cells are fibroblasts, adult stem cells, Sertoli cells, granulosa cells, neurons, pancreatic islet cells, epidermal cells, epithelial cells, endothelial cells, hepatocytes, hair follicle cells, keratinocytes, hematopoietic cells, melanocytes, chondrocytes, lymphocytes (B and T lymphocytes), macrophages, monocytes, mononuclear cells, cardiac muscle cells or skeletal muscle cells. In additional aspects, the somatic cells can be obtained by well-known methods from various organs, e.g., skin, lung, pancreas, liver, stomach, intestine, heart, reproductive organs, bladder, kidney, urethra and other urinary organs, etc., generally from any organ or tissue containing live somatic cells. In certain aspects, the precursor cells are cord blood cells, bone marrow cells, skin cells, or lymphocytes. In yet further aspects, the precursor cells are isolated from a cell population obtained from a donor, for example a human donor. For example, the cell population can be derived from cord blood, bone marrow, skin cells, or peripheral blood, for example. In some aspects, the precursor cells are lymphocytes that are isolated from cord blood, peripheral blood (e.g., from PBMCs), or bone marrow. Non-limiting examples of lymphocytes are T-lymphocytes, B-lymphocytes, and NK cells. In certain aspects, the lymphocytes are NK cells or γδ T-cells. In some aspects, the precursor cells are NK cells or γδ T-cells that are isolated from cord blood. In additional aspects, the lymphocytes are NK cells or γδ T-cells that are isolated from peripheral blood (e.g., from PBMCs). In yet further aspects, the precursor cells are NK cells or γδ T-cells that are isolated from cord blood. In additional aspects, the lymphocytes are NK cells or γδ T-cells that are isolated from bone marrow. In some aspects, the precursor cells are NK cells or γδ T-cells that are isolated from cord blood. In additional aspects, the precursor cells are CD34+ hematopoietic stem cells or CD34+ bone marrow cells. Generation of iPSCs from CD34+ cells has been described, for example, in Lee et al. (2020), Stem Cell Res. 13; 47:101913; the contents of which are expressly incorporated by reference herein. In yet further aspects, the precursor cells are primary skin cells (e.g., primary human keratinocytes), as described, for example, in Lamb et al. (2014), PLOS One 2014; 9(5): e97335, US20110143397, and WO2011123572; the contents of each of which are expressly incorporated by reference herein.

CD34+ cells, such as “CD34+ hematopoietic stem cells,” “CD34+ HSCs”, CD34+ hematopoietic progenitor cells, and “CD34+ HPCs,” and the like, express CD34. “Hematopoietic progenitor cells” or “HPCs” are cells that are capable of differentiation into blood cells such as lymphocytes, eosinophils, neutrophils, basophils, erythrocytes, and megakaryocytes. Hematopoietic progenitor cells can be recognized based on expression of CD34 and/or CD43 surface antigens. The methods described herein encompass use of a CD34+ hematopoietic progenitor cells, including hematopoietic stem cells, as a precursor cells. In additional aspects, the invention encompasses generation CD34+ HPCs from iPSCs.

The term “NK cell” or “Natural Killer cell” as well as plural referents thereof refer to a subset of peripheral blood lymphocytes defined by the expression of CD56 or CD16 and the absence of the T-cell receptor (CD3).

The term “γδ T-cell” or “gamma delta T-cell” or “gd T-cell” as well as plural referents of any of thereof refers to a subset of T-cells that express a distinct T-cell receptor (TCR). The majority of T-cells have a TCR composed of two glycoprotein chains called α- and β-TCR chains. In contrast, in γδ T-cells, the TCR is made up of one γ-chain and one δ-chain. This group of T-cells is usually much less common than αβ T-cells. γδ T-cells are unique amongst T-cell types in that they do not require stimulation via antigen processing and MHC presentation of peptide epitopes. Furthermore, γδ T-cells are believed to have a prominent role in recognition of lipid antigens, and to respond to stress-related antigens such as MIC-A and MIC-B and other ligands of the NKG2D receptor.

Human γδ T-cells can also exhibit an antigen-presenting capacity. Similar to dendritic cells (DCs), blood Vγ9Vδ2 T-cells are able to respond to signals from microbes and tumors and prime CD4⁺ and CD8⁺ T-cells. Tb T-APCs are believed to cross-present antigens directly to CD8⁺ T-cells. The intracellular protein degradation and endosomal acidification are significantly delayed in γδ T-cells in comparison to monocyte-derived DCs. The antigens are transported across IRAP (Insulin-Regulated Amino Peptidase)-positive early and late endosomes, and their processing consists of an export to the cytosol for degradation by the proteasome before being imported into an MHC-I-loading compartment. Activated γδ T-cells are able to phagocytose tumor antigens and apoptotic or live cancer cells possibly through the scavenger receptor CD36 in a C/EBPα (CCAAT/enhancer-binding protein α)-dependent mechanism and mount a tumor antigen-specific CD8⁺ T-cell response. γδ T-cells can also induce DC maturation through TNF-α production. Overall, γδ T-cells can process a wide range of antigens for presentation and stimulate other immune cells. Therefore, the γδ T-cell response to infections or cancer may be leveraged to design new strategies in order to improve clinical response of human γδ T-cell-based immunotherapy. Increased tumor immunogenicity (e.g., increased upregulation of ligands for the NKG2D receptor), e.g., resulting from a chemotherapeutic agent is uniquely conducive to γδ T-cell-mediated tumor immunosurveillance, and ultimately tumor cell killing by γδ T-cells.

A cell composition or population of cells can be enriched for the precursor cells such as immune cells, NK cells, γδ T-cells, CD34+ hematopoietic stem cells, or the engineered or genomically edited versions thereof, for example. The term “enriched,” as used herein, refers to increasing the total percentage of one or more cytotoxic immune cell types present (e.g., Tb T-cells and/or NK cells) in a sample, relative to the total percentage of the same one or more cell types prior to enrichment, as disclosed herein. For example, a sample that is “enriched” for one or more types of cytotoxic immune cell may comprise between about 10% to 100% of the one or more cytotoxic immune cell types in the sample, whereas the total percentage of one or more of the cytotoxic immune cell types in a sample prior to enrichment was, for example, between 0% and 10%. Preferably, an enriched sample comprises at least 10%, 15%, 20%, 25%, 30%, 35%, 40%, 50%, 60%, 70%, 80%, 90% or 100%, of one or more types of cytotoxic immune cell. Samples may be enriched for one or more cell types using standard techniques, for example, flow cytometry techniques. The term “highly enriched,” as used herein, refers to increasing the total percentage of one or more cytotoxic immune cell types in a sample such that the one or more cytotoxic immune cell types may comprise between at least about 70% to about 100% of the cytotoxic immune cell type in the sample, whereas the total percentage of that same type of cytotoxic immune cell prior to enrichment was, for example, between 0% and 10%. Preferably, a highly enriched sample comprises at least 60%, 65%, 70%, 75%, 80%, 85%, 90%, 95%, 99% or more of one or more types of cytotoxic immune cell. Samples may be highly enriched for one or more cell types using standard techniques, for example, flow cytometry techniques.

A “cell culture medium” (also referred to herein as a “culture medium” or “culturing medium” or “culture” or “medium”) is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

As used herein, “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

As used herein, the term “population” or “cell population” when used with reference to cells, such as a cell population isolated from a donor from which precursor cells are isolated, NK cells, or gd T-cells, refers to a group of cells including two or more of such cells. For example, the population of precursor cell derived-iPSCs can be a clonal population, in which all the cells of the population are clones of a single iPSC. In another example, the population of gd T-iPSCs can be a clonal population, in which all the cells of the population are clones of a single iPSC.

As used herein, the term “induced pluripotent stem cells” or “iPSCs,” refers to stem cells produced from differentiated adult cells or umbilical cord blood that have been induced or changed (i.e. reprogrammed) into cells capable of differentiating into tissues of all three germ or dermal layers: mesoderm, endoderm, and ectoderm. Yamanaka et al., for example, reported that transcription factors Oct3/4, Sox2, Klf4 and c-Myc confer pluripotency upon adult somatic cells and for generating iPSCs (Takahashi, K., & Yamanaka, S, Cell, 2006, 126(4):663-76; Wernig, M., et al., Nature, 2007, 448:318-324; Maherali, N., et al., Cell Stem Cell, 2007, 1(1):55-70; U.S. Pat. App. Pub. No. 20200172875; the contents of each of which are expressly incorporated by reference herein).

Precursor-cell derived induced pluripotent stem cells (iPSCs) are iPSCs produced from the isolated precursor cells, specifically including isolated human precursor cells. For example, iPSCs can be derived from umbilical cord blood cells, CD34+ HSCs, NK-cells, gd T-cells, or keratinocytes. Such cells can be referred to herein as CD34+ HSC-derived iPSCs, NK cell derived iPSCs, gd T-cell derived iPSCs, or keratinocyte derived iPSCs, respectively. In another example, iPSCs can be derived from human umbilical cord blood cells, human CD34+ HSCs, human NK-cells, human gd T-cells, or human keratinocytes. In certain aspects, the precursor cells are cord blood cells. A method of differentiating gd T cells from umbilical cord blood cell HSCs has been described for example in Boyd et al. (2021), Cells 10(10): 2631; the contents of which are expressly incorporated by reference herein. In additional aspects, the precursor cells are keratinocytes. For keratinocyte-derived iPSCs, primary skin cells can be obtained, for example, by skin biopsy and reprogrammed to iPSCs which can then be used to generate CD34+ cells, as described, for example, in Lamb et al. (2014), Broad T-Cell Receptor Repertoire in T-Lymphocytes Derived from Human Induced Pluripotent Stem Cells, PLOS One 2014; 9(5): e97335. For example, the skin cells can be transduced using a vector carrying reprogramming factors; such reprogramming factors can include Oct4, Sox2, Klf4 and c-Myc. The vector can, for example, be a lentiviral vector and the cDNAs encoding the reprogramming factors can be driven by the EF-1a promoter. In additional aspects, the precursor-cell derived iPSC is a gd T-iPSC. A “gd T-iPSC” is an iPSC that has a rearranged gd T cell receptor (TCR). When the precursor cell is other than a gd T cell, the iPSC can comprise a recombinant rearranged gd TCR. An iPSC comprising a recombinant rearranged gd TCR can prepared, for example, by genetic engineering to knock-in a set of rearranged T6 TCR transgenes. When the precursor cell is a gd T cell, the iPSC derived therefrom can comprise the rearranged gd TCR of the precursor gd T-cells (e.g., the endogenous rearranged gd TCR). Thus, the precursor cell used to generate gd T-iPSC can be a gd T-cell or a somatic cell that is not a gd T cell. The gd T-iPSCs can be derived from a precursor cell, including, but not limited, cord blood cells, bone marrow cells, skin cells, lymphocytes, or CD34+ HSCs. gd T-cell-derived iPSCs (iPSCs derived from gd T cell precursor cells) are encompassed within the terms “gd T-iPSC” and “gd T-iPSCs.” Methods of producing iPSCs from gd T-cells have been described for example in U.S. Pat. App. Pub. Nos. 20200017837, 20210395697, 20220333073, and Watanabe et al. (2018), Stem Cell Transl Med 7(1): 34-44, the contents of which are expressly incorporated by reference herein. gd T-iPSCs may express the gd T cell TCR of the parental or precursor cell. In humans, Vδ1 and Vδ2 γδ T-cells are the two main populations of γδ T-cells as based on their TCR expression. Vδ2 γδ T-cells are circulating lymphocytes and constitute the majority of peripheral blood γδ T-cells. Meanwhile, Vδ1 γδ T-cells are generally resident lymphocytes, abundant in mucosal surfaces and epithelia of the digestive, respiratory and urogenital tracts (Caron et al. (2021), Front Immunol. https://doi.org/10.3389/fimmu.2021.666983). As used herein, “gd1 T-iPSCs” are iPSCs differentiated to express the gd1 TCR. Similarly, “gd2T-iPSCs” are iPSCs differentiated to express the gd2 TCR.

The invention encompasses a cell population comprising the precursor cell-derived iPSCs prepared by a method described herein. In some examples, the precursor cell-derived iPSCs are gd T iPSCs comprising gd1 T-iPSCs and/or gd2 T-iPSCs prepared by the method described herein. In further aspects, the precursor cell-derived iPSCs are human umbilical cord blood-derived iPSCs prepared by the method described herein. In additional embodiments, the precursor cell-derived iPSCs are human keratinocyte-derived iPSCs prepared by the method described herein. In further aspects, the precursor cell-derived iPSCs are CD34+ HSC-derived iPSCs prepared by the method described herein.

The precursor cell-derived iPSCs are prepared by a process that comprises reprogramming the precursor cells into iPSCs, or in other words, engineering or editing the precursor cells to express reprogramming factors. As used herein, “reprogramming” refers to a process that alters or reverses the differentiation state of a somatic cell. The cell can be either partially or terminally differentiated prior to reprogramming. Reprogramming encompasses complete reversion of the differentiation state of a somatic cell (e.g., a T-cell) to a pluripotent state. Reprogramming also encompasses partial reversion of the differentiation state of a somatic cell to a state that renders the cell more susceptible to complete reprogramming to a pluripotent state when subjected to additional manipulations such as those described herein. Such reversion may result in expression of particular genes by the cells, which expression contributes to reprogramming. In certain embodiments, reprogramming of a somatic cell causes the somatic cell to be a pluripotent and ES-like state. The resulting cells are referred to herein as induced pluripotent stem cells (iPSCs). In some embodiments, reprogramming also encompasses partial reversion of the differentiation state of a somatic cell to a multipotent state.

Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent. In some embodiments, the methods described herein contribute to establishing the pluripotent state by reprogramming. In some embodiments, the methods described herein may be practiced on cells that are fully differentiated and/or particular types of cells (e.g., γδ T-cells), rather than on cells that are already multipotent or pluripotent.

As used herein, “reprogramming factor” refers to a gene, RNA, protein, chemical or small molecule that promotes or contributes to cell reprogramming, e.g., in vitro. Examples of reprogramming factors of interest for reprogramming somatic cells to pluripotency in vitro are Oct3/4, Klf4, c-Myc, Nanog, Sox2, and Lin28, and any gene/protein, chemical or small molecule that can substitute for one or more of these in a method of reprogramming somatic cells, e.g., in vitro. Non-limiting examples of pluripotency-associated genes (gene that encode a reprogramming transcription factor) are Oct3/4, Sox2, Nanog, Klf4, c-Myc, Nanog, Lin28, Nr5a2, Glis1, Cebpa, Esrrb, and Rex1. In some embodiments, the gene is Oct4 or Sox2.

“Reprogramming” also encompasses chemical reprogramming to produce chemically induced pluripotent stem cells (ciPSCs). For example, replacement of Oct4 with small molecule forskolin has been described (Hou et al. (2013), Science 341(6146):651-4; the contents of which are expressly incorporated by reference herein. US20200277567 (to Deng et al.) describes the use of glycogen synthase kinase inhibitors, TGFb receptor inhibitors, cyclic AMP agonists and/or histone acetylators for chemical reprogramming. Chemical reprogramming methods have also been described for example in, Liuyang et al. (2023), Cell Stem Cell 30, 1-10; Li et al., Cell Res., 21: 196 (2011); Yuan et al., Stem Cells, 29: 549 (2011); Zhu et al., Cell Stem Cell, 7: 651 (2010); Zhao et al. (2015), Cell 163(7):1678-91; US Pat App Publication No. 20210047624; US Pat App Publication No. 20200277567). Thus, the invention encompasses a method of generating functional iPSC-derived gd T-cells comprising the steps of:
a. preparing a population of precursor cell-derived induced pluripotent stem cells (iPSCs), the method comprising the steps of:
   i. isolating a population of precursor cells and culturing the precursor cells in a culturing medium, wherein the precursor cells are human somatic cells;
   ii. chemically reprogramming the precursor cells; and
   iii. culturing the reprogrammed precursor cells under conditions suitable for reprogramming the cells to pluripotency to obtain the population of precursor cell derived iPSCs; and
b. differentiating the precursor cell-derived iPSCs to CD34+ hematopoietic progenitor cells (HPCs) and generating progenitor T-cells and functional iPSC-derived gd T-cells therefrom under feeder-free conditions, wherein the iPSC-derived gd T-cells comprise gd1 T-cells and/or gd2 T-cells.

In certain embodiments, the population of precursor-cell derived iPSCs express at least one or more proteins selected from the group consisting of Oct3/4 polypeptide, a Klf4 polypeptide, a c-Myc polypeptide, a Sox2 polypeptide, a Nanog polypeptide, a Lin28 polypeptide, a Nr5a2 polypeptide, a Glis1 polypeptide, a Cebpa polypeptide, a Esrrb polypeptide, and a Rex1 polypeptide. In certain embodiments, the population of gd T iPSCs express at least one or more proteins from the group consisting of Oct3/4 polypeptide, a Klf4 polypeptide, a c-Myc polypeptide, a Sox2 polypeptide, a Nanog polypeptide, a Lin28 polypeptide, a Nr5a2 polypeptide, a Glis1 polypeptide, a Cebpa polypeptide, a Esrrb polypeptide, and a Rex1 polypeptide. In certain embodiments, the reprogramming factors used to transduce the precursor cells comprise Oct3/4, Sox2, Klf4, and c-Myc. In certain additional embodiments, the reprogramming factors used to transduce the precursor cells comprise Oct3/4, Sox2, KLF4, c-Myc, and Lin28. In certain embodiments, the reprogramming factors used to transduce the precursor cells comprise Oct3/4, Sox2, Klf4, and c-Myc.

The transduced, engineered, and/or edited precursor cells are cultured under conditions suitable for reprogramming the cells to pluripotency. As used herein, the term “pluripotent” and “pluripotency” refers to the ability of a cell to form all lineages of the body or soma (i.e., the embryo proper). For example, embryonic stem cells are a type of pluripotent stem cells that are able to form cells from each of the three germ layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

In certain embodiments, the expression vector used to introduce a pluripotency-associated gene or a reprogramming factor includes a modified viral polynucleotide, such as from an adenovirus, a Sendai virus, a herpesvirus, or a retrovirus, such as a lentiviral vector. The expression vector is not restricted to recombinant viruses and includes non-viral vectors such as DNA plasmids and in vitro transcribed mRNA. In certain aspects, the vector is a non-integrating virus vector. In particular aspects, Sendai virus vector is used. In certain embodiments, the iPSCs are negative for a Sendai virus (SeV) vector.

In additional aspects, the pluripotency-associated gene or reprogramming factor can be introduced by genome editing. Non-limiting examples of genome editing are the incorporation of a polynucleotide encoding a target gene using a transposon or using the clustered regularly interspaced short palindromic repeats (CRISPR)/Cas system.

In certain embodiments, the iPSCs are genomically stable with no loss of a chromosome. In certain embodiments, the genomic stability of the iPSCs is determined by Karyotyping analysis. In certain embodiments, the iPSCs can grow in feeder free medium after adoption.

The iPSCs are differentiated in a multi-step process to hematopoietic progenitor cells (HPCs) and then to progenitor T-cells. The term “differentiate,” “differentiation,” and the like refers to the process by which an unspecialized (or uncommitted) or less specialized cell acquires the features of a specialized cell such as, for example, a blood cell or a muscle cell. A differentiated, terminally differentiated, or differentiation-induced cell is one that has taken on a more specialized (or committed) position within the lineage of a cell. A cell is committed when it has proceeded in the differentiation pathway to a point where, under normal circumstances, it will continue to differentiate into a specific cell type or subset of cell types, and cannot, under normal circumstances, differentiate into a different cell type or revert to a less differentiated cell type.

The invention encompasses a cell population comprising CD34+ HPCs differentiated from precursor cell derived-iPSCs as described herein.

The invention further includes a cell composition comprising gd T-cells derived from CD34+ HPCs or HSCs as described herein.

The invention additionally encompasses a cell population comprising iPSC-derived gd T-cells produced by a method described herein. In certain aspects, the invention is a cell population comprising functional iPSC-derived gd T-cells comprising gd1 T cells and/or gd2 T cells. The gd T-cells can be gd1 T-cells and/or gd2 T-cells. In some embodiments, the iPSC-derived gd T-cells comprise gd1 T-cells. In additional aspects, the iPSC-derived gd T-cells comprise gd2 T-cells. In certain aspects, the gd T-cells are Vg2Vd2 T-cells, Vg9Vd2 T-cells, Vg2Vd1 T-cells, Vg3Vd1 T-cells, Vg4Vd1 T-cells, Vg5Vd1 T-cells, Vg8Vd1 T-cells, Vg9Vd1 T-cells, Vg3Vd2 T-cells, or Vg4Vd5 T-cells. In yet additional aspects, the gd T-cells, for example, the Vg2Vd2 T-cells, Vg9Vd2 T-cells, Vg2Vd1 T-cells, Vg3Vd1 T-cells, Vg4Vd1 T-cells, Vg5Vd1 T-cells, Vg8Vd1 T-cells, Vg9Vd1 T-cells, Vg3Vd2 T-cells, or Vg4Vd5 T-cells or Vg9Vd2 T-cells, comprise a transgene or exogenous polynucleotide. The transgene or exogenous polynucleotide can, for example, encode a CAR, a survival factor, or a combination thereof.

“iPSC-derived gd T-cells” are gd T-cells produced by differentiation of the precursor cell-induced pluripotent stem cells (iPSCs), for example, by differentiating the iPSCs to hematopoietic progenitor cells and progenitor T-cells. As described above, examples of precursor cell-derived iPSCs are keratinocyte-derived iPSCs, CD34+ HSC-derived iPSCs, lymphocyte-derived iPSCs, NK cell-derived iPSCs, and gd T-derived induced pluripotent stem cells (iPSCs). Methods of differentiating iPSCs, including iPSCs derived from T cells as well as non-T cells, have been described, for example, in U.S. Pat. App. Pub. Nos. 20190330596, 20190142867 and 20210130777, as well as Shukla et al. (2017), Nat Methods 14(5): 531-538, Iriguchi et al. (2021), Nat. Commun. 12: 430, and Nafria et al. (2020), STAR Protoc 1(3):100130; doi: 10.1016/j.xpro.2020.100130; the contents of each of which are expressly incorporated by reference herein. It is to be understood that the precursor-cell derived iPSCs that are differentiated to iPSC-derived gd T-cells can be iPSCs that express a survival factor and/or a CAR as described herein. The combination of specific γTCR and δTCR expressed by the iPSC-derived gd T-cells is not limited. In certain aspects, the iPSC-derived gd T-cells can, for example, have a gTCR selected from Vg1TCR, Vg2TCR, Vg3TCR, Vg4TCR, Vg5TCR, Vg6TCR, Vg7TCR, Vg8TCR and Vg9TCR and/or can have a dTCR selected from Vd1TCR, Vd2TCR, Vd3TCR, Vd4TCR, Vd5TCR, Vd6TCR, Vd7TCR, Vd8TCR and Vd9TCR. In some embodiments, the iPSC-derived gd T cells are Vd1 T cells. In yet other aspects, the iPSC-derived gd T cells are Vd2 T cells. In certain aspects, the cell population comprises Vg2Vd2 T-cells, Vg9Vd2 T-cells, Vg2Vd1 T-cells, Vg3Vd1 T-cells, Vg4Vd1 T-cells, Vg5Vd1 T-cells, Vg8Vd1 T-cells, Vg9Vd1 T-cells, Vg2Vd2 T-cells, Vg3Vd2 T-cells, Vg4Vd5 T-cells. In certain aspects, the iPSC-derived gd T-cells are Vγ2δ1 T-cells or Vγ9δ1 T-cells. In yet additional aspects, the iPSC-derived gd T-cells are Vγ2δ2 T-cells Vγ9δ2 T-cells. As discussed above, functional iPSC-derived gd T-cells are gd T-cells that have cytotoxic activity against cancer or tumor cells. The invention includes a cell population comprising the iPSC-derived gd T-cells described herein and/or prepared by a method described herein. For example, the cell population comprising the iPSC-derived gd T-cells can be differentiated from the iPSCs described herein. In yet additional aspects, the gd T-cells comprise a transgene or exogenous polynucleotide. The transgene or exogenous polynucleotide can, for example, encode a CAR, a survival factor, or a combination thereof. In certain embodiments, the cell population comprising the iPSC-derived gd T-cells comprise Vg2Vd2 T-cells, Vg9Vd2 T-cells, Vg2Vd1 T-cells, Vg3Vd1 T-cells, Vg4Vd1 T-cells, Vg5Vd1 T-cells, Vg8Vd1 T-cells, Vg9Vd1 T-cells, Vg2Vd2 T-cells, Vg3Vd2 T-cells, or Vg4Vd5 T-cells.

In some embodiments, the combination of specific 7TCR and 6TCR expressed by the iPSC-derived gd T-cells can be engineered or tailored for a specific function, specificity, and/or tropism useful for cancer therapy. Methods of expressing and/or positively selecting different gd T cell populations are described, for example, in Hahn et al. (2023), Cell Reports 42 112253; the contents of which are expressly incorporated by reference herein.

Hematopoietic progenitor cells (HPCs) can be differentiated to progenitor T-cells. Differentiation of HPCs to progenitor T-cells or gd T-lymphoid commitment is preferably performed under feeder-free conditions. The progenitor T-cells comprise cells that are CD7+/CD5−. Such cells can, for example, be prepared by culturing the HPCs in the presence of Matrigel (Niwa A, et al. PLos One, 6(7):e22261, 2011), collagen, gelatin, laminin, heparan sulfuric acid proteoglycan, RETRONECTIN®, fusion protein of DLL4 or DLL1, and Fc region of antibody (hereinafter sometimes referred to as Fc) and the like (e.g., DLL4/Fc chimera), entactin, and/or combination of these. A method of differentiating HSCs to progenitor T-cells have been described, for example, Shukla et al. Progenitor T-cell differentiation from hematopoietic stem cells using Delta-like-4 and VCAM-1. Nat Methods 14, 531-538 (2017). https://doi.org/10.1038/nmeth.4258; the contents of which are expressly incorporated by reference herein). The progenitor T-cells can then be expanded, activated, genetically modified and maturated in gd T-cells. In certain aspects, the gd T-cells can then be generated from the progenitor T-cells by activation, expansion, culture and maturation. In certain aspects, the multi-step differentiation process is conducted under feeder-free conditions and/or serum-free conditions.

“Progenitor T-cells” are cells derived from pluripotent stem cells, for example, from HPCs, and that express CD7+(human) or CD25+CD90+(mouse), and have the capacity to differentiate into one or more types of mature T-cells. A mature T-cell includes cells that express a combination of CD4, CD8 and CD3 cell surface marker. Mature T-cells include gd T-cells. The invention encompasses a cell population comprising the progenitor T-cells prepared by a method described herein, for example, progenitor T cells differentiated from precursor cell derived-iPSCs as described herein.

As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type. The feeder cells are optionally from a different species than the cells they are supporting. For example, certain types of human cells, including stem cells, can be supported by primary cultures of mouse embryonic fibroblasts, or immortalized mouse embryonic fibroblasts. In another example, peripheral blood derived cells or transformed leukemia cells support the expansion and maturation of natural killer cells. The feeder cells may typically be inactivated when being co-cultured with other cells by irradiation or treatment with an anti-mitotic agent such as mitomycin to prevent them from outgrowing the cells they are supporting. Feeder cells may include endothelial cells, stromal cells (for example, epithelial cells or fibroblasts), and leukemic cells. Without limiting the foregoing, one specific feeder cell type may be a human feeder, such as a human skin fibroblast. Another feeder cell type may be mouse embryonic fibroblasts (MEF). In general, various feeder cells can be used in part to maintain pluripotency, direct differentiation towards a certain lineage, enhance proliferation capacity and promote maturation to a specialized cell type, such as an effector cell.

As used herein, a “feeder-free” (FF) environment or “feeder-free” conditions refers to an environment such as a culture condition, cell culture or culture media which is essentially free of feeder cells or stromal cells.

The methods described herein can comprise a step of identifying reprogrammed mammalian somatic cells or confirming that reprogramming has occurred. Methods for identifying reprogrammed mammalian somatic cells having a less differentiated state or a pluripotent state are known in the art. For example, in some embodiments, reprogrammed somatic cells are identified by selecting for cells that express the appropriate selectable marker. In some embodiments, reprogrammed somatic cells are further assessed for pluripotency characteristics. The presence of pluripotency characteristics indicates that the somatic cells have been reprogrammed to a pluripotent state. Thus, to assess reprogrammed somatic cells for pluripotency characteristics, one may analyze such cells for different growth characteristics and ES cell-like morphology. In some embodiments, cells may be injected subcutaneously into immunocompromised SCID mice to induce teratomas (a standard assay for ES cells). ES-like cells can be differentiated into embryoid bodies (another ES specific feature). Moreover, ES-like cells can be differentiated in vitro by adding certain growth factors known to drive differentiation into specific cell types. Self-renewing capacity, marked by induction of telomerase activity, is another pluripotency characteristics that can be monitored. If the reprogrammed cells are capable of forming a few cell types of the body, they are multipotent; if the reprogrammed cells are capable of forming all cell types of the body including germ cells, they are pluripotent. In other embodiments, the expression of an individual pluripotency gene in the reprogrammed somatic cells may be examined to assess their pluripotency characteristics. Additionally, one may assess the expression of other ES cell markers. Stage-specific embryonic antigens-1, -3, and -4 (SSEA-1, SSEA-3, SSEA-4) are glycoproteins specifically expressed in early embryonic development and are markers for ES cells (Solter and Knowles, 1978, Proc. Natl. Acad. Sci. USA 75:5565-5569; Kannagi et al., 1983, EMBO J 2:2355-2361).

Differentiation status of cells is a continuous spectrum, with terminally differentiated state at one end of this spectrum and de-differentiated state (pluripotent state) at the other end. Reprogramming, as used herein, refers to a process that alters or reverses the differentiation status of a somatic cell, which can be either partially or terminally differentiated. Reprogramming includes complete reversion, as well as partial reversion, of the differentiation status of a somatic cell. In other words, the term “reprogramming,” as used herein, encompasses any movement of the differentiation status of a cell along the spectrum toward a less-differentiated state. For example, reprogramming includes reversing a multipotent cell back to a pluripotent cell, reversing a terminally differentiated cell back to either a multipotent cell or a pluripotent cell. In one embodiment, reprogramming of a somatic cell turns the somatic cell all the way back to a pluripotent state. In another embodiment, reprogramming of a somatic cell turns the somatic cell back to a multipotent state. The term “less-differentiated state,” as used herein, is thus a relative term and includes a completely de-differentiated state and a partially differentiated state.

The invention encompasses a method of generating functional iPSC-derived gd T-cells comprising the steps of preparing a population of induced pluripotent stem cells (iPSCs) and differentiating the iPSCs to CD34+ hematopoietic progenitor cells (HPCs) and generating progenitor T-cells and the functional iPSC-derived gd T-cells therefrom under feeder-free conditions. In certain aspects, the iPSCs are human keratinocyte-derived iPSCs and the iPSCs are differentiated to CD34+CD34+ hematopoietic progenitor cells (HPCs) and progenitor T-cells are generated, and the functional iPSC-derived gd T-cells therefrom. In additional aspects, the iPSCs are CD34+ hematopoietic stem cell-derived iPSCs and the iPSCs are differentiated to CD34+CD34+ hematopoietic progenitor cells (HPCs), progenitor T-cells are generated, and the functional iPSC-derived gd T-cells therefrom. In yet additional aspects, the iPSCs are gdT iPSCs, optionally wherein the population of the gdT iPSCs comprises gd1T-iPSCs and/or gd2T-iPSCs, and differentiating the gd T iPSCs to CD34+ hematopoietic progenitor cells (HPCs) and generating progenitor T-cells and the functional iPSC-derived gd T-cells therefrom under feeder-free conditions. Preparation of the gd T iPSCs can comprise the steps of: i) activating and expanding lymphocytes, such as NK cells or T-lymphocytes, from a cell population isolated from a donor wherein the activation and expansion comprises culturing the cell population (such as, cord blood cells, bone marrow cells, peripheral blood cells (e.g., PBMCs), CD34+ HSCs, or keratinocytes) in a culturing medium under conditions suitable for activation and expansion; ii) transducing the precursor cells with a non-integrating virus vector encoding a plurality of reprogramming factors, optionally comprising Oct3/4, Sox2, Kfl4 and c-Myc; and iii) culturing the transduced precursor cells under conditions suitable for reprogramming the cells to pluripotency to obtain the population of gd T-iPSCs. As described above, the cell population isolated from a donor can be derived from cord blood, bone marrow, or peripheral blood, for example. In yet another example, the cell population are PBMCs. In another example, the precursor cells are skin cells or keratinocytes. The precursor cells can, for example, be obtained from a donor, for example, a human donor.

The invention also encompasses a method of generating functional iPSC-derived gd T-cells comprising the steps of preparing a population of induced pluripotent stem cells. The iPSCs can be derived from cord blood cells, CD34+ HSCs, lymphocytes, or human keratinocytes. In certain aspects, the iPSCs are gd T iPSCs. The gd T iPSCs can, for example, comprise gd1T-iPSCs and/or gd2T-iPSCs. The methods encompass, differentiating the iPSCs, for example, the gd T iPSCs, to CD34+ hematopoietic progenitor cells (HPCs) and generating progenitor T-cells and the functional iPSC-derived gd T-cells therefrom under feeder-free conditions. Preparation of the gd T iPSCs can comprise the steps of: i) activating and expanding gd T-cells from a cell population isolated from a donor wherein the activation and expansion comprises culturing the cell population (such as, cord blood cells, bone marrow cells or peripheral blood cells) in a culturing medium under conditions suitable for activation and expansion; ii) engineering the precursor cells to express plurality of reprogramming factors, optionally comprising Oct3/4, Sox2, Kfl4 and c-Myc; and iii) culturing the transduced precursor cells under conditions suitable for reprogramming the cells to pluripotency to obtain the population of gd1 T and/or gd2 T-iPSCs. In certain aspects, preparation of the gd T iPSCs can comprise the steps of: i) activating and expanding gd T-cells from a cell population isolated from a donor wherein the activation and expansion comprises culturing the cell population (such as, cord blood cells, bone marrow cells or, peripheral blood cells) in a culturing medium under conditions suitable for activation and expansion; ii) transducing the precursor cells with a non-integrating virus vector encoding a plurality of reprogramming factors, optionally comprising Oct3/4, Sox2, Kfl4 and c-Myc; and iii) culturing the transduced precursor cells under conditions suitable for reprogramming the cells to pluripotency to obtain the population of gd1 T and/or gd2 T-iPSCs. The invention encompasses a cell population comprising gd T iPSCs prepared by the method described herein. In yet additional aspects, preparation of the iPSCs can comprise the steps of: (i) transducing CD34+ HSCs with a non-integrating virus vector encoding a plurality of reprogramming factors, optionally comprising Oct3/4, Sox2, Kfl4 and c-Myc; and iii) culturing the transduced precursor cells under conditions suitable for reprogramming the cells to pluripotency to obtain the population of gd1 T and/or gd2 T-iPSCs. In yet further embodiments, preparation of the iPSCs can comprise the steps of: (i) transducing primary human keratinocytes with a non-integrating virus vector encoding a plurality of reprogramming factors, optionally comprising Oct3/4, Sox2, Kfl4 and c-Myc; and iii) culturing the transduced precursor cells under conditions suitable for reprogramming the cells to pluripotency to obtain the population of gd1 T and/or gd2 T-iPSCs. In yet further aspects, the CD34+ HSC-derived iPSCs or the keratinocyte-derived iPSCs are engineered to express a rearranged gd TCR. The invention encompasses a cell population comprising the iPSCs prepared by the method described herein. As described above, the precursor cell population isolated from a donor can be derived from cord blood, bone marrow, or peripheral blood, for example. In yet other examples, the precursor cells are skin cells or keratinocytes. In yet another example, the cell population isolated from a donor are PBMCs.

A non-limiting example of a non-integrating virus vector is Sendai virus.

The terms “expanded” and “expansion” as used herein with regard to expansion of one or more cytotoxic immune cells, for example, gd T-cells, in a sample means to increase in the number of one or more cytotoxic immune cells in the sample by, for example about at least about 2-fold, at least about 5-fold, at least 10-fold, at least 50-fold, at least about 100-fold, or at least about 1000-fold, or more. Expansion of a cytotoxic immune cell population can be accomplished by any number of methods as are known in the art. For example, T-cells can be rapidly expanded using non-specific T-cells receptor stimulation in the presence of feeder lymphocytes and interleukin-. Methods of activating and expanding γδ T-cells have been described, for example, in WO2017035375 and WO2011053750; the contents of which are expressly incorporated by reference herein. Methods for isolating γδ T-cells either from a patient to be treated or from another source, are described, for example, in U.S. Pat. No. 7,078,034, incorporated herein by reference in its entirety. For example, gd T-cells can be activated and expanded by contacting a cell population such as cord blood cells, bone marrow cells, skin cells or PBMCs with an appropriate culturing medium (e.g., in activation culture). In certain aspects, the gd T-cells are activated and expanded from PBMCs. The culturing medium can comprise an interleukin (for example, IL-2, IL-15, and/or IL-23) and an antigen or agent that simulates expansion of gd T cells. Such antigens or agents include, but are not limited to, isopentenyl pyrophosphate (IPP) (22), (E)-4-hydroxy-3-methyl-but-2-enyl pyrophosphate (HMB-PP) zoledronate (Zometa) and bromohydrin pyrophosphate (BrHPP) (Wang et al. (2019), Mol Med Rep 19(3): 1471-1480). The culturing medium, for example, comprises IL-2 and a bisphosphonate. Bisphosphonates include, but are not limited to, zolendronic acid or zolendronate, pamidronic acid, alendronic acid, risedronic acid, ibandronic acid, incadronic acid, etidronic acid, minodronic acid, salts thereof and hydrates thereof. In additional aspects, gd T-cells can be activated and expanded by contacting the PBMCs with bromohydrin pyrophosphate (BrHPP) and IL2 (see, for example, Chargui et al. (2010), J Immunother 33(6): 591-8). In additional aspects, the culturing medium for Vd1 cells can comprise a cytokine such as IL-7, phytohemagglutinin (PHA), anti-CD2, anti-CD3, pan-d antibodies (such as monoclonal antibodies), and anti-Vd1 antibodies (such as monoclonal antibodies), or a combination of any of thereof (as described for example, in Wu et al. (2015), OncoImmunulogy 4:3, e992749; Siegers et al. (2014), Mol. Ther. 22(8): 1416-1422; the contents of which are expressly incorporated by reference herein.

As described above, the precursor cells can be obtained from a donor. Alternatively, the precursor cells can be isolated from a cell population obtained from a donor. Examples of precursor cells include cord blood cells, bone marrow cells, skin cells (including keratinocytes), lymphocytes, and CD34+ HSCs. In certain aspects, the precursor cells are cord blood cells. In additional aspects, the precursor cells are bone marrow cells. In yet further aspect, the precursor cells are skin cells. In yet additional aspects, the precursor cells are NK cells. In further aspects, the precursor cells are human skin cells including primary human keratinocytes. In yet additional aspects, the precursor cells are CD34+ HSCs. In certain additional aspects, the precursor cells are gd T-cells. The culturing medium for activation and expansion of the gd T-cells can, for example, comprise IL-2 and an agent selected from a bisphosphonate and a phosphoantigen. A non-limiting example of a bisphosphonate is zolendronic acid or zolendronate. A non-limiting example of a phosphoantigen is bromohydrin pyrophosphate (BrHPP) or isopentenyl pyrophosphate (IPP). In another example, the culturing medium can comprise a cytokine such as IL-7, phytohemagglutinin (PHA), anti-CD2, anti-CD3, pan-d antibodies (such as monoclonal antibodies), and anti-Vd1 antibodies (such as monoclonal antibodies), or a combination of any of thereof.

As described above, the gdT-iPSCs, including the gdT-derived iPSCs, have a rearranged gdT TCR locus. The rearrangement can be confirmed with genotype sequencing. The methods described herein include methods of preparing gd1 T-iPSCs and/or gd2 T-iPSCs. The gd1 T-iPSCs can, for example, have the Vγ2-to-Jγ1/2 and Vd1-to-Jd1 recombination or the Vγ4-to-Jγ1/2 and Vd1-to-Jd1 to recombination. The gd2 T-iPSCs can, for example, have the Vγ2-to-Jγ1/2 and Vd2-to-Jd1 recombination. The gd T-iPSCs can express one or more reprogramming factors. For example, at least about 90%, at least about 92%, or at least about 95% of the gd1T-iPSCs can be OCT3/4 and SSEA4 double positive. In another example, at least about 90%, at least about 92%, or at least about 95% of the gd2T-iPSCs can be OCT3/4 and SSEA4 double positive. In addition, the gd T-iPSCs can be confirmed as having a normal karyotype. The gd T-iPSCs are functional, for example, when the gdT iPSCs have cytotoxic activity against tumor or cancer cells, for example, leukemia cell lines, glioma, and/or glioblastoma tumor cells. In certain aspects, gd T-iPSCs are also phenotypically normal in that they express the cell surface markers (for example, detected using flow cytometry) that define gd T-cells. In further aspects the gd T-iPSCs express a polypeptide selected from the group consisting of a chimeric antigen receptor (CAR), a polypeptide the confers resistance to a chemotherapeutic agent, as well as a combination thereof. In further aspects the gd1T-iPSCs or gd2T-iPSCs are transduced or edited, for example, with a transgene or polynucleotide encoding a polypeptide selected from the group consisting of a chimeric antigen receptor (CAR), a polypeptide the confers resistance to a chemotherapeutic agent, as well as a combination thereof. In certain aspects, the progenitor T-cells are activated and expanded. In further examples, the progenitor T-cells are thereafter activated, expanded, and maturated. In certain aspects, the CD34+ HPCs are differentiated from a clonal master gd T-iPSC population. The clonal gd T iPSC population can be gd1 T-iPSCs or gd2 T-iPSCs.

The invention also encompasses a population of precursor cell-derived iPSCs, lymphocyte-derived iPSCs, NK-cell derived iPSCs, gd T-cell derived-iPSCs, keratinocyte-derived iPSCs, CD34+ HSC-derived iPSCs, a population of CD34+ hematopoietic progenitor cells (HPCs), a population of progenitor T-cells and a population of functional iPSC-derived gd T-cells, wherein the foregoing are prepared by a method described herein. In further aspects, the iPSCs express a polypeptide selected from the group consisting of a chimeric antigen receptor (CAR), a survival factor, and a combination thereof. In yet further aspects, the gdT-iPSC comprise a transgene or a polynucleotide that encodes a polypeptide selected from the group consisting of a chimeric antigen receptor (CAR), a survival factor, and a combination thereof. In specific aspects, the invention is a population of gd1 T-iPSCs or functional iPSC-derived gd1 T-cells. In additional aspects, the invention is a population of gd2 T-iPSCs or functional iPSC-derived gd2 T-cells. In further aspects, the iPSC-derived gd T-cells express a polypeptide selected from the group consisting of a chimeric antigen receptor (CAR), a survival factor, and a combination thereof. In additional aspects, the iPSC-derived gd T-cells, express a transgene that encodes a polypeptide selected from the group consisting of a chimeric antigen receptor (CAR), a survival factor, and a combination thereof.

As described herein, the precursor cell-derived iPSCs can be engineered to express a polypeptide selected from the group consisting of a chimeric antigen receptor (CAR), and survival factor, and a combination thereof.

Methods of engineering (e.g., to express a CAR, a survival factor or other gene or polypeptide) include, for example, transfection, viral infection, viral vectors, AAV vectors, and genome editing. Techniques that can be used to introduce a transgene into a target cell are well known in the art. For instance, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. Additional techniques include squeeze-poration, lipofection, laserfection, sonoporation, and the use of microvesicles. A variety of vectors for the delivery and integration of polynucleotides encoding exogenous proteins into the nuclear DNA of a mammalian cell have been developed. Viral genomes are particularly useful vectors for gene delivery because the polynucleotides contained within such genomes are typically incorporated into the nuclear genome of a mammalian cell by generalized or specialized transduction. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, 1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses.

Various editing technologies are known, and include CRISPR, zinc fingers (ZFs) and transcription activator-like effectors (TALENs). Fusion proteins containing one or more of these DNA-binding domains and the cleavage domain of Fokl endonuclease can be used to create a double-strand break in a desired region of DNA in a cell. Exemplary techniques are described, for example, in US Patent Appl. Pub. No. US 2012/0064620, US Patent Appl. Pub. No. US 2011/0239315, U.S. Pat. No. 8,470,973, US Patent Appl. Pub. No. US 2013/0217119, U.S. Pat. No. 8,420,782, US Patent Appl. Pub. No. US 2011/0301073, US Patent Appl. Pub. No. US 2011/0145940, U.S. Pat. Nos. 8,450,471, 8,440,431, 8,440,432, and US Patent Appl. Pub. No. 2013/0122581, the contents of all of which are hereby incorporated by reference). In some embodiments, gene editing is conducted using CRISPR associated Cas system, as known in the art. See, for example, U.S. Pat. Nos. 8,697,359, 8,906,616, and 8,999,641, which is hereby incorporated by reference in its entirety. Genome editing techniques specifically include the integration of target genes into the genome of a target cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can design a CRISPR/Cas system to cleave any target DNA molecule of interest. This technique has been exploited in order to edit eukaryotic genomes (Hwang et al., Nature Biotechnology 31:227 (2013)) and can be used as an efficient means of site-specifically editing target cell genomes in order to cleave DNA prior to the incorporation of a gene encoding a target gene. The use of CRISPR/Cas to modulate gene expression has been described in, for example, U.S. Pat. No. 8,697,359, the disclosure of which is incorporated herein by reference as it pertains to the use of the CRISPR/Cas system for genome editing. Additional specific examples of CRISPR/Cas systems and methods that can be used for genome editing are described, for example, in U.S. Pat. Pub. No. 20190144888 and U.S. Pat. Pub. No. 20190032089; the contents of each of which are expressly incorporated by reference herein. An additional technique includes an alternative CRISPR nuclease referred to as MAD7 that has been disclosed in U.S. Pat. Nos. 9,982,279 and 10,337,028, the contents of which are expressly incorporated herein. The company Inscripta has made this nuclease-free for all commercial or academic research. Inscripta reports that MAD7 was developed from Eubacterium rectale and has proven its functionality in E. coli, S. cerevisiae and in the human HEK293T cell line. MAD7 has only 31% identity with Acidaminococcus sp. BV3L6 Cpf1 (AsCpf1), to which it also shares a T-rich PAM site (5′-YTTN-3′), and a protospacer (the region of the gRNA which associates the nuclease to the DNA target) length of 21 nucleotides. Yet additional examples of editing systems and methods are carried out using ZFN, TALEN, homing nuclease, homology recombination, or any other functional variation of these systems or methods.

As discussed herein, the precursor cell derived iPSCs (for example, the gd T iPSCs), the iPSC-derived gd T cells, or the CD34+ HSC-derived gd T cells can be engineered to express a survival factor. In certain specific aspects, the iPSC-derived gd T-cells express a survival peptide. In additional aspects, the precursor cell-derived iPSCs are engineered to express a survival factor. The invention encompasses a cell population comprising iPSC-derived gd T cells that express a survival factor as well as precursor cell derived iPSCs that express a survival factor. gd T-cells that express a survival factor can be used in methods of drug resistant immunotherapy as described, for example, U.S. Pat. No. 10,322,145, the contents of which are expressly incorporated by reference herein. The term “drug resistant immunotherapy” or DRI is a strategy for treating cancer whereby anti-cancer immune cells, preferably γδ T-cells, are genetically engineered to resist the toxic effects of chemotherapy drugs which allows for the combined administration of chemotherapy and immunotherapy. Chemotherapy resistance or the acquisition of chemoresistance is a well-known phenomenon in the field of cancer treatment. Such resistance to chemotherapeutic agents can arise from the expression of certain DNA, RNA or polypeptides that impact drug resistance genes, expression of a gene that conveys drug resistance, the expression of a polypeptide that confers resistance to chemotherapeutic agents. The DRI strategy described herein uses chemoresistance to confer resistance to the immune cells that can be used in cancer immunotherapy. DRI precursor cell-derived iPSCs, for example, include iPSCs that have been genetically engineered to express a survival factor as described herein, including, but not limited to, a DNA, RNA or polypeptide that confers resistance to a chemotherapeutic agent. DRI iPSC-derived gd T-cells, for example, include iPSC-derived gd T-cells that express or have been genetically engineered to express a survival factor as described herein, including, but not limited to, a DNA, RNA or polypeptide that confers resistance to a chemotherapeutic agent. A polypeptide that confers resistance to a chemotherapeutic agent can be referred to herein as a “survival polypeptide” or a “survival factor”).

The term “survival factor” refers to any agent now known or later discovered in the art that confers resistance to a chemotherapeutic agent, and/or to a chemotherapeutic agent treatment regimen and/or allows the cells comprising the survival factor to survive in a treatment environment (such as a chemotherapy treatment environment). Exemplary survival factors have been described, for example, in U.S. Pat. Nos. 10,322,145 and 10,543,233 as well as WO2018/035413, WO2017041106 and WO2018107134, the contents of each of which are expressly incorporated by reference herein. The phrase “confers resistance” and the like encompasses the acquisition of resistance to a chemotherapeutic agent or improvement in resistance to a chemotherapeutic agent. The “survival factor” includes an agent that confers resistance to a chemotherapeutic agent when it is expressed by the γδ T-cell. The “survival factor” can thus be a DNA, RNA or polypeptide that is expressed by the γδ T-cells (e.g., encoded by a drug resistance gene) and that confers resistance to a chemotherapeutic agent. As described herein, the γδ T-cell can be engineered to express the DNA, RNA or polypeptide that confers resistance to a chemotherapeutic drug by including a vector which expresses a gene, a gene fragment, a DNA, an siRNA, or an mRNA, that encodes the survival factor that confers resistance to a chemotherapeutic agent. In yet other aspects, the survival factor is a DNA that confers resistance to a chemotherapeutic agent. In further aspects, the survival factor is an RNA (e.g., a RNAi, siRNA, microRNA, or mRNA) that confers resistance to a chemotherapeutic agent.

In certain aspects, the survival factor is a polypeptide that confers resistance to a chemotherapeutic agent; for example, the polypeptide confers resistance when it is expressed by the γδ T-cells. The survival polypeptide or polypeptide that confers resistance to a chemotherapeutic agent can be any polypeptide known in the art that provides resistance to a treatment regimen comprising a chemotherapeutic agent, and/or allows the cells comprising the survival polypeptide to survive in a treatment environment created by the chemotherapeutic agent. Exemplary chemotherapeutic agents are nucleoside-analog chemotherapy drug, alkylating agent, antimetabolite, antibiotic, topoisomerase inhibitor, mitotic inhibitor, differentiating agent, or hormone therapy agent and the survival factor provides resistance to the chemotherapeutic agent. In additional aspects, the chemotherapeutic agent is an alkylating agent. In certain embodiments, the survival polypeptide is MGMT, multidrug resistance protein 1 (MDR1), or 5′ nucleotidase II (NT5C2). In yet further aspects, the survival polypeptide is MGMT and the chemotherapeutic agent is an alkylating agent such as carmustine (BCNU), lomustine (CCNU), and temozolomide. In certain aspects, the chemotherapeutic agent is temozolomide (TMZ). In additional aspects, the survival polypeptide is MDR1 and the chemotherapeutic agent is an anthracycline, vinca alkaloids, epipodophyllotoxins, camptothecin, methotrexate (MTX), saquinavir, and mitoxantrone (MX) (Sodani et all. (2011). Multidrug resistance associated proteins in multidrug resistance. Chin J Cancer 31(2): 58-72). NT5C2 is a polypeptide known in the art to provide resistance to thiopurine chemotherapy (Tzoneva et al. (2013), Activating mutations in the NT5C2 nucleotidase gene drive chemotherapy resistance in relapsed ALL, Nat Med. 19(3): 368-371). Other survival polypeptides include, for example, a drug resistant variant of dihydrofolate reductase (L22Y-DHFR) and thymidylate synthase. In certain aspects, the survival polypeptide is MGMT. However, other survival factors may be used depending on the chemotherapeutic agent being co-administered, the nature of the treatment environment (i.e., what other treatment regimens are being given to the patient in combination with the cells compositions of the present disclosure). In certain embodiments, the survival factor is MGMT, multidrug resistance protein 1 (MDR1), or 5′ nucleotidase II (NT5C2). Other survival factors include, for example, a drug resistant variant of dihydrofolate reductase (L22Y-DHFR) and thymidylate synthase. In certain aspects, the survival factor is MGMT. Other polypeptides that confer resistance may be used or expressed by the cell depending on the nature of the treatment environment (i.e., what other treatment regimens are being given to the patient in combination with the cells compositions of the present disclosure). MGMT repairs alkylating lesions of the DNA by removing mutagenic adducts from the 06 position of guanine. Such mutagenic adducts can be caused by alkylating agents (including, but not limited to, temozolomide). Thus, MGMT is a polypeptide that confers resistance to alkylating agents such as temozolomide. The survival factor can be a polypeptide that confers resistance to a chemotherapeutic agent, including, but not limited to, the specific chemotherapeutic agents described herein.

In additional aspects, the chemotherapeutic agent to which the survival factor confers resistance is an alkylating agent; a metabolic antagonist; a DNA demethylating agent; a substituted nucleotide; a substituted nucleoside; an antitumor antibiotic; a plant-derived antitumor agent or a nitrosourea. Preferably the chemotherapeutic agent is selected from cisplatin; carboplatin; cyclophosphamide; etoposide; fludarabine; methotrexate (MTX); trimethotrexate (TMTX); temozolomide; dacarbazine (DTIC), raltitrexed; S-(4-Nitrobenzyl)-6-thioinosine (NBMPR); 6-benzyguanidine (6-BG); a nitrosourea (rabinopyranosyl-N-methyl-N-nitrosourea (Aranose), Carmustine (BCNU, BiCNU), Chlorozotocin, Ethylnitrosourea (ENU), Fotemustine, Lomustine (CCNU), Nimustine, N-Nitroso-N-methylurea (NMU), Ranimustine (MCNU), Semustine, Streptozocin (Streptozotocin)); cytarabine; camptothecin; and a therapeutic derivative of any thereof. Preferably, the γδ T-cells have been genetically modified to encode alkyl guanine transferase (AGT), P140K-MGMT, O⁶ methylguanine DNA methyltransferase (MGMT), L22Y-DHFR, thymidylate synthase, dihydrofolate reductase, or multiple drug resistance-1 protein (MDR1). In additional examples, the γδ T-cells can be genetically modified to be resistant to at least two chemotherapeutic agents selected from: an alkylating agent; a metabolic antagonist; a DNA demethylating agent; a substituted nucleotide; a substituted nucleoside; an antitumor antibiotic; a plant-derived antitumor agent and a nitrosurea. In some embodiments, the γδ T-cells are genetically modified to be resistant to at least two chemotherapeutic agents selected from cisplatin; carboplatin; etoposide; methotrexate (MTX); trimethotrexate (TMTX); temozolomide; dacarbazine (DTIC), raltitrexed; S-(4-Nitrobenzyl)-6-thioinosine (NBMPR); 6-benzyguanidine (6-BG); a nitrosourea (rabinopyranosyl-N-methyl-N-nitrosourea (Aranose), Carmustine (BCNU, BiCNU), Chlorozotocin, Ethylnitrosourea (ENU), Fotemustine, Lomustine (CCNU), Nimustine, N-Nitroso-N-methylurea (NMU), Ranimustine (MCNU), Semustine, Streptozocin (Streptozotocin)); cytarabine; camptothecin; and a therapeutic derivative of any thereof. Preferably, the chemotherapeutic agent is TMZ, methotrexate, DTIC, BCNU, CCNU, MCNU, NMU or ENU. In additional aspects, the chemotherapeutic agent includes, but is not limited to: alkylating agents (e.g., cyclophosphamide, ifosfamide, melphalan); metabolic antagonists (e.g., methotrexate (MTX), 5-fluorouracil or derivatives thereof); DNA demethylating agents (also known as antimetabolites; e.g., azacitidine): a substituted nucleotide; a substituted nucleoside; antitumor antibiotics (e.g., mitomycin, adriamycin); plant-derived antitumor agents (e.g., vincristine, vindesine, TAXOL®, paclitaxel, abraxane); cisplatin; carboplatin; etoposide; and the like. Such agents may further include, but are not limited to, the anti-cancer agents trimethotrexate (TMTX); temozolomide (TMZ); raltitrexed; S-(4-Nitrobenzyl)-6-thioinosine (NBMPR); 6-benzyguanidine (6-BG); nitrosoureas (for example, bis-chloroethylnitrosourea, also known as BCNU and carmustine, lomustine, also known as CCNU, +/− procarbazine and vincristine (PCV regimen) and fotemustine); doxorubicin; cytarabine; camptothecin; and a therapeutic derivative of any thereof.

As discussed herein, the precursor cell derived iPSCs (for example, the gd T iPSCs), the iPSC-derived gd T cells, or the CD34+ HSC-derived gd T cells is engineered to express a chimeric antigen receptor (CAR). The invention encompasses a cell population comprising iPSC-derived gd T cells that express a CAR as well as precursor cell derived iPSCs that express a CAR. The term “chimeric antigen receptor(s)” and “CAR(s)),” as used herein, refers to artificial T-cell receptors, T-bodies, single-chain immunoreceptors, chimeric T-cell receptors, or chimeric immunoreceptors, for example, and encompass engineered receptors that graft an artificial specificity (for example, an antigen recognition domain) onto a particular immune effector cell, for example, □□ T-cells. In some embodiments, CARs comprise an intracellular activation domain, a transmembrane domain, and an extracellular domain that may vary in length and that comprises an antigen recognition domain. Also as described herein, a CAR can lack an intracellular signaling domain, for example, the CD3z intracellular signaling domain. Exemplary CARs have been described, for example, in WO2017041106, WO2018107134 and WO2022159582 (PCT Application No. PCT/US22/13130), the contents of which are expressly incorporated by reference herein. This disclosure encompasses precursor cell-derived iPSCs that express a CAR, gd T iPSCs that express a CAR, as well as iPSC-derived gd T-cells that express a CAR.

The terms “antigen recognition domain,” “antigen recognition moiety,” “antigen binding domain,” “antigen binding moiety,” and the like, are used interchangeably herein. Similarly, the terms, “transmembrane domain,” “transmembrane moiety,” “transmembrane region,” and the like are used interchangeably; the terms “hinge domain,” “hinge moiety,” and “hinge region,” and the like are used interchangeably; the terms “intracellular signaling domain,” “signaling domain,” “signaling moiety,” “signaling region,” and the like are used interchangeably herein.

In certain aspects, the CAR is directed to a tumor antigen or a tumor associated antigen (TAA). In one aspect of the foregoing, the tumor antigen is any tumor antigen known in the art. In certain embodiments, the tumor antigen is selected so at least one of the following characteristics is present: the antigen is expressed in as many stages of the cancer as possible, the antigen is expressed on the surface of the tumor, the antigen is important to the viability of the tumor cell and the antigen is not expressed on non-tumor tissue or expressed at such a level that off target effects are clinically acceptable. In certain embodiments, the tumor antigen is selected from the group consisting of EphA2, B cell maturation antigen (BCMA), B7-H3, B7-H6, CAIX, CA9, CD22, CD19, CD20, ROR1, kappa or light chain, carcinoembryonic antigen, alpha-fetoprotein, CA-125, Glypican-3, epithelial tumor antigen, melanoma-associated antigen, EGP2, EGP40, EPCAM, ERBB3, ERBB4, ErbB3/4, PAP, FAR, FBP, fetal AchR, Folate Receptor a, mutated p53, mutated ras, HER2, ERBB2, HER3, folate binding protein, HIV-1 envelope glycoprotein gp120, HIV-1 envelope glycoprotein gp41, 5T4, 8H9, GD2, CD123, CD23, CD33, CD30, CD38, CD56, c-Met, fap, mesothelin, GD3, HERV-K, IL-2Ra, IL-24b, IL-13Ra, CSPG4, Lewis-Y, MCSP, Mucl, Mucl6, NCAM, NKG2D ligands, NY-ESO-1, PRAME, PSCA, PSC1, PSMA, EGFR, Spl7, SURVIVFN, TAG72, TEM1, TEM8, EGFRvIII, and VEGFR2.

In additional examples, the CAR is chlorotoxin (CLTX) CAR. As used herein, the terms “chlorotoxin” and “CLTX” or “CTX” are used interchangeably and refer to a scorpion venom peptide, chlorotoxin, that comprises 36 amino acids having the amino acid sequence: MCMPCFTTDHQMARKCDDCCGGKGRGKCYGPQCLCR (SEQ ID NO: 1) (UniProt Accession #P45639). Without wishing to bound by theory, the CLTX peptide binding domain can act to enhance trafficking of the gd T-cells to solid tumors including, but not limited to, gliomas, liver cancer, ovarian cancers and others that express the target. The CLTX peptide can also enhance trafficking to melanoma, small cell lung carcinoma, neuroblastoma, breast cancer, kidney cancer, liver cancer, lung cancer, ovarian cancer, and medulloblastoma. A CAR comprising a CLTX peptide within its antigen recognition domain is referred to herein as a CLTX-CAR. CLTX-CARs are described, for example, in WO2018107134 and WO2022159582 (PCT Application No. PCT/US22/13130), the contents of which are expressly incorporated by reference herein. A CLTX-CAR can comprise one or more CLTX peptides. CLTX peptides include single CLTX-CARs, dual CLTX-CARs and other multivalent CLTX-CARs. The term “dual CLTX-CAR” or “dCLTX-CAR” or “divalent CAR” or “2×CLTX-CAR” refers to a CAR comprising two CLTX peptides in the antigen recognition domain; the two CLTX peptides can be attached by a linker peptide. A “single CLTX-CAR” or “sCLTX-CAR” or “1×CLTX-CAR” refers to a CAR comprising only one CLTX peptide in the antigen recognition domain. In multivalent CLTX-CARs, the antigen recognition domain can comprise more than one CLTX peptide, for example, two, three, four, five, or more CLTX peptides. A CAR comprising at least one CLTX peptide within its antigen recognition domain is referred to herein as a CLTX-CAR. A “multivalent CLTX-CAR” is a CTLX-CAR that comprises more than one CLTX peptide in the antigen recognition domain; for example, the more than one CLTX peptide can be linked by a peptide or peptides. Such linking peptides are referred to herein as the “linker peptide” or the “linking peptide.” The linker peptide that links a pair of CLTX peptides can be the same or different from the peptide that links a different pair of CLTX peptides in the antigen recognition domain; for example, in a multivalent CLTX-CAR comprising three CLTX peptides, the linker peptide that links two peptides (e.g., the first and the second peptide) can be the same or different from the linker peptide that links (e.g., the second peptide and the third peptide).

The specificity of other CAR designs may be derived from ligands of receptors (e.g., peptides). In certain cases, the spacing of the antigen-recognition domain can be modified to reduce activation-induced cell death. In certain cases, the intracellular signaling domain of the CARs comprise domains for additional co-stimulatory signaling, such as, but not limited to, FcR, CD27, CD28, CD 137, DAP 10, and/or OX40 in addition to CD3ζ. In some cases, molecules can be co-expressed with the CAR, including co-stimulatory molecules, reporter genes for imaging (e.g., for positron emission tomography), gene products that allow host-cells expressing the CAR to survive in a treatment environment created by an additional therapeutic treatment, gene products that conditionally ablate the host-cells expressing the CAR upon addition of a pro-drug, homing receptors, chemokines, chemokine receptors, cytokines, and cytokine receptors.

Also as described herein are embodiments in which the CAR gd T-cell does not comprise an intracellular signaling domain. The intracellular signaling domain of the CAR described herein is responsible for activation of at least one of the normal effector functions of the host-cell in which the CAR is placed. The term “effector function” refers to a specialized function of a differentiated cell. When the host-cell is an immune effector cell, the intracellular signaling domain of the CAR of the invention is responsible for activation of at least one of a normal immune effector function. Immune effector function of a T-cell, for example, may be cytolytic activity or helper activity, including, but not limited to, the secretion of cytokines. Thus, the term “intracellular signaling domain” refers to the portion of a CAR that transduces the effector function signal and directs the cell to perform a specialized function (for example, an effector function and/or an immune effector function). While usually the entire intracellular signaling domain will be employed, in many cases, it will not be necessary to use the entire intracellular signaling domain. To the extent that a truncated portion of the intracellular signaling domain may find use, such truncated portion may be used in place of the intact signaling domain as long as such truncated portion still transduces the effector function/immune effector function signal. The term “intracellular signaling domain” is thus meant to include any truncated portion of the intracellular signaling domain sufficient to transduce the effector function/immune effector function signal. Examples of intracellular signaling domains include, but are not limited to, a signaling domain from the zeta chain of the T-cell receptor (CD3 zeta; CD247) or any of its homologs (e.g., eta, delta, gamma, or epsilon), MB1 chain, B29, FcRIII, FcRI, and combinations of signaling and/or costimulatory molecules, such as CD3 zeta chain and CD28, CD27, 4-IBB, DAP-10, OX40, and combinations thereof, as well as other similar molecules and fragments as well as mutations to the foregoing, such as modifying the immunoreceptor tyrosine-based activation motif(s) (ITAMs). In certain embodiments, the signaling domain comprises a CD3 zeta sequence, which may be represented by the sequence: RVKFSRSADAPAYQQGQNQLYNELNLGRREEYDVLDKRRGRDPEMGGKPRRKNP QEGLYNELQKDKMAEAYSEIGMKGERRRGKGHDGLYQGLSTATKDTYDALHMQ ALPPR (SEQ ID NO: 2). Intracellular signaling portions of other members of the families of activating proteins can be used, such as FcyRIII and FcRI. One of skill in the art will be able to determine the corresponding signaling domains. Furthermore, any of the signaling domain sequences may contain from 1 to 5 amino acid modifications, which may be selected as discussed herein. In certain aspects, the endodomain does not include an intracellular signaling domain.

In certain aspects, the CAR comprises a sequence encoding a costimulatory signaling domain. For example, the endodomain can comprise a sequence encoding a primary signaling domain and a sequence encoding a costimulatory signaling domain. In certain embodiments, the costimulatory domain is a functional signaling domain from 41BB, OX40 and/or CD28.

The extracellular domain comprising the antigen recognition domain can be linked to the intracellular signaling domain via an extracellular spacer (also referred to herein as an extracellular hinge domain) and/or a transmembrane domain. The extracellular antigen binding domain and the transmembrane domain can be linked by an extracellular hinge domain or an extracellular spacer sequence. Preferably, the extracellular spacer or extracellular hinge domain sequence comprises one or more of a hinge region and/or a portion of an immunoglobulin heavy chain constant region (which may comprise CH1, a linker region, CH2 and/or CH3 domains) or any combination thereof, of a human immunoglobulin, i.e., IgA, IgD, IgE, IgG, and IgM. In certain embodiments, extracellular spacer or hinge domain comprises all or a portion of the hinge region of human IgD. In certain embodiments, extracellular spacer or hinge comprises all or a portion of the hinge region of human IgG1. In certain embodiments, the extracellular spacer or hinge comprises all or a portion of the hinge region of human IgD and all or a portion of the hinge region of human IgG1. In certain embodiments, the extracellular spacer or hinge comprises all or a portion of the hinge region of human IgD and all or a portion of the CH2 and CH3 domains of the heavy-chain constant region of human IgG1. In certain embodiments, the extracellular spacer or hinge comprises all or a portion of the hinge region of human IgD, all or a portion of the hinge region of human IgG1 and all or a portion of the CH2 and CH3 domains of the heavy chain constant region of human IgG1. In certain embodiments, the extracellular spacer or hinge comprises all or a portion of the hinge region of human IgG1 and all or a portion of the CH2 and CH3 domains of the heavy chain constant region of human IgG1. In certain embodiments, extracellular spacer or hinge comprises all of the hinge region of human IgD, all or a portion of the hinge region of human IgG1 and the heavy chain constant region comprises all or a portion of the CH2 and CH3 domains of human IgG1. Preferably the hinge region amino acid sequence comprises the hinge region amino acid sequence from an immunoglobulin, such from IgD or IgG1, wherein the amino acid sequence comprises from 1 to 5 amino acid modifications, which may be selected as discussed herein. Preferably, the CH2 and CH3 domains of the heavy chain constant region comprises the CH2 and CH3 domain immunoglobulin heavy chain constant region amino add sequence from an immunoglobulin, such from IgG 1, wherein the amino acid sequence comprises from 1 to 5 amino acid modifications, which may be selected as discussed herein. In other aspects, the extracellular spacer or the extracellular hinge domain comprises the hinge region of a protein selected from the group consisting of CD8a, CD28, CD137, or a combination thereof. In certain aspects, the extracellular spacer or the extracellular hinge domain comprises the hinge region of CD8a. In any of the foregoing, the extracellular spacer may further comprise a linker, such as a linker having the sequence of Ser-Gly-Gly-Gly (SEQ ID NO: 3) or Ser-Gly-Gly-Gly-Gly (SEQ ID NO: 4), which may be present having from 1 to 10 copies, linking the extracellular spacer to the extracellular antigen binding domain.

In certain embodiments, the antigen recognition domain is linked to the transmembrane domain via a flexible linker. The flexible linker may be present in addition to the extracellular spacer or instead of the extracellular space described herein. In certain embodiments, the extracellular domain/antigen recognition domain is linked to the extracellular spacer via a flexible linker. Preferably, the flexible linker comprises, for example, glycine and serine. Preferably, the flexible linker is comprised of a polypeptide having the sequence of SEQ ID NO: 5 (Ser-Gly-Gly-Gly)ₙ or SEQ ID NO: 4 (Ser-Gly-Gly-Gly-Gly) wherein n is an integer from 1 to 10. Preferably, each flexible linker is a polypeptide comprising from about 1-25 amino acids, preferably about 1-15 amino acids, preferably about 1-10 amino acids, preferably about 4-24 amino acids, preferably about 5-20 amino acids, preferably about 5-15 amino acids and preferably about 5-12 amino acids. Preferably, the linker is (Ser-Gly-Gly-Gly)ₙ(SEQ ID NO: 5) wherein n is 3.

The CAR described herein can comprise a transmembrane domain that corresponds to, or is derived or obtained from, the transmembrane domain of any molecule known in the art. For example, the transmembrane domain can correspond to that of a CD8 molecule or a CD28 molecule. CD8 is a transmembrane glycoprotein that serves as a co-receptor for the T-cell receptor (TCR) and is expressed primarily on the surface of cytotoxic T-cells. The most common form of CD8 exists as a dimer composed of a CD8 and CD8P chain. CD28 is expressed on T-cells and provides co-stimulatory signals required for T-cell activation. A transmembrane domain from a CD8 polypeptide may have the sequence IYIWAPLAGTCGVLLLSLVITLYC (SEQ ID NO: 6), particularly amino acids 1-21, 1-23 or 1-24). CD28 is the receptor for CD80 (B7.1) and CD86 (B7.2). A transmembrane domain from a CD28 polypeptide may have the sequence FWVLVVVG GVLACYSLLVTVAFIIFWV (SEQ ID NO: 7). Preferably, the CD8 and CD28 are human. Preferred transmembrane domains of the CARs described herein include, but are not limited to, all or a portion of a transmembrane domain from a polypeptide selected from: an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDIIa, CD18), ICOS (CD278), 4-IBB (CD137), GITR, CD40, BAFFR, HVEM (LIGHTR), SLAMF7, NKp80 (KLRFl), CD160, CDI9, IL2R□, 1L2Rγ, IL7R□, ITGA1, VLA1, CD49a, ITGA4, IA4, CD49D, ITGA6, VLA-6, CD49f, ITGAD, CDIId, ITGAE, CD 103, ITGAL, CD1 la, LFA-1, ITGAM, CDllb, ITGAX, CD1 lc, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD 160 (BY55), PSGL1, CD100 (SEMA4D), SLAMF6 (NTB-A, Ly108), SLAM (SLAMF1, CD 150, IPO-3), BLAME (SLAMF8), SELPLG (CD 162), LTBR, PAG/Cbp, NKp44, NKp30, NKp46, NKG2D, and/or NKG2C. One of skill in the art will be able to determine the corresponding transmembrane regions from these polypeptides.

The CAR can comprise any one of the aforementioned transmembrane domains and any one or more (e.g., 1, 2, 3, or 4) of the aforementioned intracellular T-cell signaling domains in any combination and any of the aforementioned hinge domain and any of the aforementioned co-stimulatory domains. For example, the CAR can comprise a CD28 transmembrane domain and intracellular T-cell signaling domains of CD28 and CD3zeta. Furthermore, any of the transmembrane domain sequences may contain from 1 to 5 amino acid modifications, which may be selected as discussed herein.

An exemplary CAR of the invention can comprise an antigen recognition domain comprising a CAR directed to a tumor antigen or a CLTX-CAR. The CAR can further comprises one or more of the following: an optional linker linking the antigen recognition domain to the hinge domain; a hinge domain comprising all or a portion of a hinge region of CD8a, CD28, or CD137; preferably, the hinge region of CD8a; a transmembrane region from CD28; and/or an optional intracellular signaling region (or endodomain) comprising at least one signaling domain; preferably, CD3zeta, and an optional costimulatory signaling domain as described herein; preferably, the CD28 and/or the 4-1BB co-stimulatory domains. In yet further aspects, the CAR, for example, the CLTX-CAR can comprise an extracellular signal peptide. For example, the signal peptide can be the signal peptide of a protein selected from the group consisting of CD8a, CD28, GM-CSF, CD4, CD137, or a combination thereof.

In certain aspects, the CLTX-CAR comprises only one CLTX peptide or only two CLTX peptides in the antigen binding domain.

In certain aspects, the CAR does not comprise or include an intracellular signaling domain. Such a CAR can comprise an extracellular domain (also referred to herein as an “ectodomain”) comprising an antigen recognition domain/moiety. In yet further non-limiting examples, the CAR can further comprise a hinge domain and a transmembrane domain. In yet additional embodiments, the CAR does not comprise or include a CD3 zeta signaling domain. The multivalent CAR that does not comprise or include an intracellular signaling domain may or may not include a co-stimulatory domain.

This disclosure explicitly includes γδ T-cells and gd T-iPSCs that are engineered to express a CAR and a survival factor, wherein the survival factor is a DNA, RNA, or polypeptide that confers resistance to a chemotherapeutic agent. This disclosure also includes a cell population comprising γδ T-cells that express a CAR and a survival factor and/or gd T-iPSCs that are engineered to express a CAR and a survival factor, wherein the survival factor is a DNA, RNA, or polypeptide that confers resistance to a chemotherapeutic agent. The invention additionally provides an engineered γδ T-cell that expresses a chimeric antigen receptor (CAR) and a polypeptide that confers resistance to a chemotherapeutic agent, wherein the γδ T-cell comprises a single vector that directs the expression of the CAR and the polypeptide that confers resistance to a chemotherapeutic agent. The CAR can be directed to a tumor antigen and/or a chlorotoxin CAR as described herein. As described above, the use of the survival factor, including, for example, the polypeptide that confers resistance to a chemotherapeutic agent (such as the MGMT polypeptide), enables the compositions comprising the engineered □□ T-cells of the present disclosure (including a DR γδ T-cells) to survive in a treatment environment created by the chemotherapeutic agent at a time when the tumor is stressed. The stress effect on the tumor (e.g., by the chemotherapeutic agent) in certain embodiments increases the expression of stress antigens, which are recognized by receptors, such as the NKG2D receptor, on the γδ T-cells. The dual effect of inducing stress antigens and decreasing regulatory T-cells with chemotherapy significantly improve tumor reduction over either individual regimen. Gene modification and genome editing (expressing the survival polypeptide) and/or treatment with a survival factor as described herein protects the compositions of the present disclosure from the lymphodepleting effects of a chemotherapy regimen, for example TMZ, and allows the cell compositions of the present disclosure specific access to the tumor via TAA combined with unimpaired T-cell cytotoxic function at the time that malignant-cells are maximally stressed by chemotherapy. The use of DRI in combination with a CAR in accordance with the invention is referred to herein as “DRI CAR” therapy, is believed to significantly prolong survival and reduce tumor burden and time to recurrence when compared with either chemotherapy (for example, TMZ) treatment alone or γδ T-cell infusion, for example, alone and do so without significant adverse systemic or neurologic consequences.

The engineered CAR γδ T cell as described herein can optionally further express a suicide gene. A “suicide gene” as used herein refers to a mechanism by which the CAR-expressing cells described herein may be eradicated from a subject administered with the cells or a composition thereof, for example, in order to protect against a cascading inflammatory response or off-target cytotoxicity. The suicide gene system can, for example, be a Herpes Simplex Virus Thymidine Kinase (HSVTK)/Ganciclovir (GCV) suicide gene system, an inducible Caspase suicide gene system (Budde et al., PLoS One 2013 8(12):82742), codon-optimized CD20 (Marin et al., Hum. Gene Ther. Meth. 2012 23(6)376-86), CD34, a truncated EGFR (Wang X, Chang W-C, Wong C W, et al. A transgene-encoded cell surface polypeptide for selection, in vivo tracking, and ablation of engineered cells. Blood. 2011; 118(5):1255-1263. doi:10.1182/blood-2011-02-337360), a truncated CD19, or polypeptide RQR8 (Philip et al, and WO2013153391A, which is hereby incorporated herein by reference). An additional example of a suicide gene is the r-retrovirus SFG.iCaspase9.2A.DeltaCD19 which consists of iC9 linked, via a 2A-like sequence, to truncated human CD 19 that serves as selectable marker. AP1903-inducible activation of the Caspase 9 suicide gene is achieved by expressing a chimeric protein (iC9), fused to a drug-binding domain derived from human FK506-binding protein (FKBP). The iC9 is quiescent inside cells until exposure to API 903, which cross-links the FKBP domains, initiates iCasp9 signaling, and induces apoptosis of the gene-modified cells. The gene and API 903 is available from Bellicum Pharmaceuticals (Houston, TX).

The iPSC-derived γδ T-cells, a population of such cells, or a pharmaceutical composition comprising the cells can be utilized in the treatment of cancer or tumor. Specifically, the methods of treatment described herein comprise administration of the γδ T-cells as described herein and optionally, with a chemotherapeutic agent, can be used to reduce a cancer or tumor. The terms “reducing a cancer,” “inhibition of cancer,” “inhibiting cancer,” “preventing cancer recurrence,” and similar terms and are used interchangeably herein and refer to one or more of a reduction in the size or volume of a tumor mass, a decrease in the number of metastasized tumors in a subject, a decrease in the proliferative status (the degree to which the cancer cells are multiplying) of the cancer cells, prevention of recurrences of previous tumors or the development of new metastases, and the like. The term “reducing a tumor” as used herein refers to a reduction in the size or volume of a tumor mass, a decrease in the number of metastasized tumors in a subject, a decrease in the proliferative status (the degree to which the cancer cells are multiplying) of the cancer cells, and the like. The methods of treatment described herein can comprise administration of the iPSC-derived γδ T-cells with a chemotherapeutic agent can be used to reduce a tumor. For example, the methods of treatment can comprise co-administration of iPSC-derived γδ T-cells that express a survival factor or a polypeptide that confers resistance to a chemotherapeutic with a chemotherapeutic agent. In another example the methods of treatment can comprise co-administration of iPSC-derived γδ T-cells that express a CAR, such as a CAR directed to a tumor antigen or a chlorotoxin CAR. In yet further examples, the methods of treatment can comprise co-administration of iPSC-derived γδ T-cells that express a survival factor or a polypeptide that confers resistance to a chemotherapeutic with a chemotherapeutic agent and that express a express a CAR, such as a CAR directed to a tumor antigen or a chlorotoxin CAR. As used herein, any form of administration of a “combination”, “combined therapy” and/or “combined treatment regimen,” or “co-administration” or “co-administering,” or the like, refers to administration of at least two therapeutically active drugs or compositions (e.g., administration of the γδ T-cells and chemotherapeutic agent, or pharmaceutical compositions thereof), simultaneously or substantially simultaneously in either separate or combined formulations, or sequentially at different times separated by minutes, hours, days, weeks, or months, but in some way act together to provide the desired therapeutic response, for example, as part of the same treatment regimen.

The term “chemotherapeutic agent” as used herein refers to a compound or a derivative thereof that can interact with a cancer cell, thereby reducing the proliferative status of the cell and/or killing the cell for example, by impairing cell division or DNA synthesis, or by damaging DNA, effectively targeting fast dividing cells. Examples of chemotherapeutic agents include, but are not limited to, alkylating agents (e.g., cyclophosphamide, ifosfamide, temozolomide); metabolic antagonists (e.g., methotrexate (MTX), 5-fluorouracil or derivatives thereof); a substituted nucleotide; a substituted nucleoside; DNA demethylating agents (also known as antimetabolites; e.g., azacitidine); antitumor antibiotics (e.g., mitomycin, adriamycin); plant-derived antitumor agents (e.g., vincristine, vindesine, TAXOL®, paclitaxel, abraxane); cisplatin; carboplatin; etoposide; and the like. Such agents may further include, but are not limited to, the anti-cancer agents trimethotrexate (TMTX); temozolomide (TMZ); raltitrexed; S-(4-Nitrobenzyl)-6-thioinosine (NBMPR); 6-benzyguanidine (6-BG); a nitrosoureas a nitrosourea (rabinopyranosyl-N-methyl-N-nitrosourea (Aranose), Carmustine (BCNU, BiCNU), Chlorozotocin, Ethylnitrosourea (ENU), Fotemustine, Lomustine (CCNU), Nimustine, N-Nitroso-N-methylurea (NMU), Ranimustine (MCNU), Semustine, and Streptozocin (Streptozotocin)); cytarabine; and camptothecin; or a therapeutic derivative of any thereof.

The γδ T-cells and/or the chemotherapeutic agent and/or additional agent used in the treatment method are used in an effective amount or a therapeutically effective amount. The phrase “therapeutically effective amount” or an “effective amount” in the context of the administration of an agent or composition to a subject, refers to an amount capable of having any detectable, positive effect on any symptom, aspect, or characteristic of a disease, disorder or condition, when administered to the subject; the agent or composition can be administered either alone or as part of a pharmaceutical composition and either in a single dose or as part of a series of doses. The therapeutically effective amount or effective amount can be ascertained by measuring relevant physiological effects, and it can be adjusted in connection with the dosing regimen and diagnostic analysis of the subject's condition, and the like. In reference to cancer or pathologies related to unregulated cell division, a therapeutically effective amount or an effective amount can refer to that amount which has the effect of (1) reducing the size of a tumor (i.e. tumor regression), (2) inhibiting (that is, slowing to some extent, preferably stopping) aberrant-cell division, for example cancer cell division, (3) preventing or reducing the metastasis of cancer cells, (4) relieving to some extent (or, preferably, eliminating) one or more symptoms associated with a pathology related to or caused in part by unregulated or aberrant-cellular division, including for example, cancer, (5) increasing the survival or life expectancy of the subject, and/or (6) decreasing the risk of relapse. An “effective amount” is also that amount that results in desirable PD and PK profiles and desirable immune cell profiling upon administration of the therapeutically active compositions of the invention. An “effective amount” is also an amount that achieves a recited effect or result; for example, an effective amount a chemotherapeutic agent that, alone or when in combination with another agent, can be an amount that reduces the size of a tumor and/or increases stress antigen expression on the tumor cells, and/or has a cytotoxic effect.

The terms “treating” or “treatment” of a disease (or a condition or a disorder) as used herein refer to inhibiting the disease (slowing or arresting its development), providing relief from the symptoms or side-effects of the disease (including palliative treatment), preventing or delaying recurrence, and causing regression of the disease. With regard to cancer, these terms also mean that the life expectancy of an individual affected with a cancer may be increased or that one or more of the symptoms of the disease will be reduced. With regard to cancer, “treating” also includes enhancing or prolonging an anti-tumor response in a subject.

The terms “subject” and “patient” as used herein include humans, mammals (e.g., cats, dogs, horses, etc.), living cells, and other living organisms. The “donor” from whom a cell population or precursor cells are obtained can be a subject or a patient. A living organism can be a mammal. Typical patients are mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Preferably, a system includes a sample and a subject. The term “living host” refers to host or organisms noted above that are alive and are not dead. The term “living host” refers to the entire host or organism and not just a part excised (e.g., a liver or other organ) from the living host. In preferred aspects, the subject or patient is a human subject or patient.

By “administration” is meant introducing a compound, biological materials including a cell population, or a combination thereof, or a composition comprising any of the aforementioned compounds, biological materials (e.g., a cell population), or a combination thereof, of the present invention into a human or animal subject. One preferred route of administration of the compounds is intravenous. Another preferred route is parenteral. “Parenteral” refers to a route of administration that is associated with injection, including intraorbital, infusion, intraarterial, intracapsular, intracardiac, intradermal, intramuscular, intraperitoneal, intrapulmonary, intraspinal, intrasternal, intrathecal, intracranial, intrauterine, intravenous, subarachnoid, subcapsular, subcutaneous, transmucosal, or transtracheal. Other exemplary routes of administration of the compounds may be intraperitoneal or intrapleural, or via a catheter to the brain. However, any route of administration, such as oral, topical, subcutaneous, peritoneal, intra-arterial, inhalation, vaginal, rectal, nasal, introduction into the cerebrospinal fluid, introduction into a vein (such as hepatic portal vein to deliver to liver), intracranial, ocular, or instillation into body compartments can be used. Direct injection into a target tissue site such as a solid tumor is also contemplated. For example, intracranial administration of the γδ T-cells for the treatment of a glioma or other intracranial tumor can be used.

The term “cancer,” as used herein, shall be given its ordinary meaning, as a general term for diseases in which abnormal cells divide without control. Cancer cells can invade nearby tissues and can spread through the bloodstream and lymphatic system to other parts of the body. When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors. Carcinoma is cancer that begins in the skin or in tissues that line or cover internal organs. Glioma is a tumor that arises from the supportive (“gluey”) tissue of the brain, called glia, which helps to keep the neurons in place and functioning well. Sarcoma is cancer that begins in bone, cartilage, fat, muscle, blood vessels, or other connective or supportive tissue. Leukemia is cancer that starts in blood-forming tissue such as the bone marrow and causes large numbers of abnormal blood cells to be produced and enter the bloodstream. Lymphoma is cancer that begins in the cells of the immune system.

Representative cancers include, but are not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Glioblastoma, Childhood; Glioblastoma, Adult; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet-cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chrome Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ. Cell Tumor, Childhood; Extragonadal Germ. Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hvpopharyngeal Cancer; Hypothalamic and Visual Pathway Glioma, Childhood; Intraocular Melanoma; Islet-cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chrome Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chrome; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neuroendocrine Tumor; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Ocular Melanoma; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood, Pancreatic Cancer, Islet-cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland' Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a different environment, not normally conducive to their growth), and continue their rapid growth and division in this new location. This is called metastasis. Once malignant-cells have metastasized, achieving a cure or treatment is more difficult. Benign tumors have less of a tendency to invade and are less likely to metastasize.

The cancer to be treated can be of neuroectodermal origin. In certain aspects, the cancer is a malignant glioma, melanoma, neuroblastoma, medulloblastoma or small cell lung carcinoma. The infused cells are able to kill tumor cells in the recipient. The invention also includes a cellular therapy where γδ T cells are modified to transiently express a CAR of the invention and the survival polypeptide, wherein the cells are infused to a recipient in need thereof.

The infused cells are able to kill tumor cells in the recipient. Thus, in various aspects, the γδ T cells administered to the patient, is present for less than one month, e.g., three weeks, two weeks, one week, after administration to the patient. The method encompasses repeat dosing, for example, administration at one more intervals of about one week, administration at one or more intervals of about two weeks, or administration at one or more intervals of about a month. In certain aspects, the cells administered to the patient, or their progeny, persist in the patient for at least four months, five months, six months, seven months, eight months, nine months, ten months, eleven months, twelve months, thirteen months, fourteen months, fifteen months, sixteen months, seventeen months, eighteen months, nineteen months, twenty months, twenty-one months, twenty-two months, twenty-three months, two years, three years, four years, five years, or ten years, or more, after administration of the host-cells to the patient.

In further aspects, iPSC-derived γδ T-cells can be used in a type of vaccine for ex vivo immunization and/or in vivo therapy in a mammal. In one aspect, the mammal is a human.

The iPSC-derived γδ T-cells and a chemotherapeutic agent (e.g., the chemotherapeutic agent to which the survival factor confers resistance) can be co-administered. Such co-administration can encompass “simultaneous” or “concurrent delivery,” e.g., in the same or in separate compositions. In other aspects, co-administration encompasses separate administration but as part of the same treatment regimen. In certain aspect, the chemotherapeutic agent is administered before or concurrently with the γδ T-cells. In additional aspects, the γδ T-cells are co-administered with the chemotherapeutic agent, wherein the chemotherapeutic agent causes increased expression of a stress ligand (e.g., NKG2DL) on the tumor or cancer cells; for example, the chemotherapeutic agent is administered in an amount and in a manner/regiment resulting in increased express of the stress ligands. In certain aspects, the co-administration can be more effective than that of either treatment alone. The effect of the two treatments can be partially additive, wholly additive, or greater than additive. For example, co-administration can encompass administration of the γδ T-cells about 1 hour to about 72 hours, about 2 hours to about 72 hours, about 4 hours to about 72 hours, about 6 hours to about 72 hours, or about 8 hours to about 72 hours after administration of the chemotherapeutic agent. In certain aspects, the γδ T-cells are administered about 12 hours to about 36 hours after administration of the chemotherapeutic agent; for example, the γδ T-cells are administered about 24 hours after administration of the chemotherapeutic agent. In yet further aspects, co-administration encompasses administering the γδ T-cells at the same time or at substantially the same time as the chemotherapeutic agent. As used herein “substantially the same time” can encompass administration within the same treatment session.

The iPSC-derived γδ T-cells and the chemotherapeutic agent can be administered during periods of active disorder, suspected recurrence, or during a period of remission or less active disease.

In further aspects, an additional therapeutic agent is administered in addition to the iPSC-derived γδ T-cells and optionally, the chemotherapeutic agent. When administered in combination, the γδ T-cells and the chemotherapeutic agent and/or the additional therapeutic agent, the amount or dosage of one or all of the foregoing, can be administered in an amount or dose that is higher, lower or the same than the amount or dosage of each agent used individually, e.g., as a monotherapy. In certain embodiments, the amount or dosage of one or all of the foregoing, is lower (e.g., at least 20%, at least 30%, at least 40%, or at least 50%) than the amount or dosage of each agent used individually, e.g., as a monotherapy. In other embodiments, the amount or dosage of one or all of the foregoing, that results in a desired effect (e.g., inhibition of cancer) is lower (e.g., at least 20%, at least 30%, at least 40%, or at least 50% lower) than the amount or dosage of each agent used individually, e.g., as a monotherapy, required to achieve the same therapeutic effect.

The γδ T-cells and optionally, the chemotherapeutic agent, can be administered in combination with an additional therapeutic treatment, such as, but not limited to, surgery, chemotherapy (e.g., an additional chemotherapeutic agent different from the chemotherapeutic agent to which the DR cells are resistant), checkpoint inhibitors, PARP inhibitors, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxan, fludarabine, FK506, rapamycin, mycophenolic acid, steroids, and cytokines. In yet additional aspects, the additional therapeutic agent is an immune checkpoint inhibitor, as described, for example, in WO2018/035413, the contents of which are expressly incorporated by reference herein. For example, immune checkpoint inhibitor can be one that targets CTLA-4, PDL1, PDL2, PD1, B7-H3, B7-H4, BTLA, HVEM, TIM3, GAL9, LAG3, VISTA, KIR, 2B4, CD160 (also referred to as BY55), CGEN-15049, CHK 1 kinase, CHK2 kinase, A2aR, OX40, or a B-7 family ligand. In further aspects, the additional therapeutic agent is a DDR inhibitor, including but not limited to PARP inhibitors as described, for example, in WO 2020/097306, the contents of which are expressly incorporated by reference herein. Non-limiting examples of PARP inhibitors are olaparib, rucaparib, niraparib, Talazoparib (Pfizer), veliparib (Abbvie), E7016 (Eisai), CEP-9722 (Teva), and BGB-290 (Pamiparib, BeiGene), as well as combinations thereof.

The therapies disclosed herein can be administered to patient by various routes including, for example, parenterally and can include but not be limited to, intravenously, intramuscularly, subcutaneously, intraorbitally, intracapsularly, intraperitoneally, intrarectally, intracisternally, intratumorally, intravasally, intradermally, intravaginally (e.g., vaginal suppositories), or topically (e.g., powders, ointments transdermal patch) or by passive or facilitated absorption through the skin using, for example, a skin patch or transdermal iontophoresis, respectively.

Preferably, the total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the composition to treat a pathologic condition in a subject depends on many factors including the age and general health of the subject as well as the route of administration and the number of treatments to be administered. In view of these factors, the skilled artisan would adjust the particular dose, as necessary.

The cell compositions described herein can be delivered as a pharmaceutical composition, or made into an implant appropriate for administration in vivo, with appropriate carriers or diluents, which further can be pharmaceutically acceptable. The means of making such a composition or an implant have been described in the art. Where appropriate, the γδ T-cells described herein can be formulated into a preparation in semisolid or liquid form, such as a solution, injection, inhalant, or aerosol, in the usual ways for their respective route of administration. Means known in the art can be utilized to prevent or minimize release and absorption of the composition until it reaches the target tissue or organ, or to ensure timed-release of the composition. Desirably, however, a pharmaceutically acceptable form is employed which does not ineffectuate the cells. Thus, desirably the cells as described herein can be made into a pharmaceutical composition containing a balanced salt solution, for example, Hanks' balanced salt solution, or normal saline. Therefore, the invention includes pharmaceutical compositions comprising γδ T-cells of the present disclosure, and specifically includes γδ T-cells expressing a CAR and/or expressing the polypeptide that confers resistance to a polypeptide.

The pharmaceutical composition can be used alone or in combination with other well-established agents useful for treating cancer, for example, a chemotherapeutic agent as described herein. Whether delivered alone or in combination with other agents, the pharmaceutical composition of the present invention can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

The composition can be provided in unit dosage form wherein each dosage unit, e.g., an injection, contains a predetermined amount of the composition, alone or in appropriate combination with oilier active agents. The term unit dosage form as used herein refers to physically discrete units suitable as unitary dosages for human and animal subjects, each unit containing a predetermined quantity of the composition of the present invention, alone or in combination with other active agents, calculated in an amount sufficient to produce the desired effect, in association with a pharmaceutically acceptable diluent, carrier, or vehicle, where appropriate. The specifications for the novel unit dosage forms of the present invention depend on the particular pharmacodynamics associated with the pharmaceutical composition in the particular subject. Preferably, a therapeutically effective amount or sufficient number of the engineered γδ T-cells, administered alone or in combination with a therapeutic agent, is introduced into the subject such that a long-term, specific, response is established. In one embodiment, the response includes inhibition of cancer. In one embodiment, the response is the reduction in size of a tumor or elimination of tumor growth or regrowth or a reduction in metastasis to a greater degree than would otherwise result in the absence of the treatment with the γδ T-cells or composition thereof. In certain aspects, the therapeutically effective amount results in at least about 5%, 10%, 20%, 30%, 40%, 50%, 60%, 70%, 80%, 90%, 95%, 98%, or 100% decrease in tumor size when compared that in the absence of the γδ T-cells or composition thereof. Accordingly, the therapeutically effective amount takes into account the route of administration and the number of engineered cells should be such that a sufficient number of so as to achieve the desired therapeutic response. Furthermore, the amounts of γδ T-cells of the present disclosure or other cell included in the compositions described herein (e.g., the amount per each cell to be contacted or the amount per certain body weight) can vary in different applications. The dosing schedule can be based on well-established cell-based therapies or an alternate continuous infusion strategy can be employed.

These amounts provide general guidance to be utilized by the practitioner upon optimizing the method of the present invention for practice of the invention. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on inter-individual differences in pharmacokinetics, drug disposition, and metabolism. One skilled in the art readily can make any necessary adjustments in accordance with the exigencies of the particular situation. Suitable doses for a therapeutic effect would be between about 10⁵ and about 10¹⁰ host-cells per dose, preferably in a series of dosing cycles. Suitable modes of administration include intravenous, subcutaneous, intracavitary (for example by reservoir-access device), intraperitoneal, and direct injection into a tumor mass.

The pharmaceutical compositions of the invention can be formulated to be compatible with the intended method or route of administration; exemplary routes of administration are set forth herein. Furthermore, the pharmaceutical compositions can be used in combination with other therapeutically active agents or compounds as described herein in order to treat or prevent the diseases, disorders and conditions as contemplated by the present disclosure.

The pharmaceutical compositions typically comprise a therapeutically effective amount of one or more agents used in the combination therapies of the invention and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle can be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it can be stored, for example, in bags, sterile vials as a solution, or suspension. Such formulations can be stored either in a ready-to-use form, a lyophilized form requiring reconstitution prior to use, a liquid form requiring dilution prior to use, or other acceptable form. The pharmaceutical composition is provided in a single-use container (e.g., a single-use vial, ampoule, syringe, or autoinjector (similar to, e.g., an EPIPEN®), whereas a multi-use container (e.g., a multi-use vial) is provided in other embodiments. The composition can be delivered by injection, pump, or other appropriate device. Any drug delivery apparatus can be used to deliver the cell population, including implants (e.g., implantable pumps) and catheter systems, slow injection pumps and devices, all of which are well known to the skilled artisan.

The pharmaceutical compositions can be in the form of a sterile injectable aqueous or oleaginous suspension. This suspension can be formulated according to the known art using those suitable dispersing or wetting agents and suspending agents mentioned herein. The sterile injectable preparation can also be a sterile injectable solution or suspension in a non-toxic parenterally-acceptable diluent or solvent, for example, as a solution in 1,3-butane diol. Acceptable diluents, solvents and dispersion media that can be employed include water, Ringer's solution, isotonic sodium chloride solution, CREMOPHOR EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil can be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The pharmaceutical compositions suitable for use in accordance with the invention may be in any format currently known or developed in the future.

The treatment methods described herein are particularly suitable for the treatment of cancer. Cancer cells can invade nearby tissues and can spread through the bloodstream and lymphatic system to other parts of the body. There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow and causes large numbers of abnormal blood cells to be produced and enter the bloodstream. Lymphoma is cancer that begins in the cells of the immune system.

The cancer to tumor being treated can be an intracranial tumor. Intracranial tumors include, but are not limited to, gliomas, meningiomas, acoustic neuromas, pituitary adenomas, medulloblastomas, germ cell tumors and craniopharyngiomas. In some aspects, the cancer being treated in accordance with the invention is a CNS tumor including, but not limited to, intracranial and spinal ependymoma (excluding subependymoma); low grade infiltrative supratentorial astrocytoma/oligodendroglioma, medulloblastoma, anaplastic gliomas, glioblastoma, metastatic lesion of the CNS and primary CNS lymphoma. In some aspects, the cancer being treated is melanoma. Preferably the cancer being treated is uveal melanoma. In some aspects, the cancer being treated is a neuroendocrine or adrenal tumor. Examples include but are not limited to bronchopulmonary disease, GI tract, lung or thymus, pancreas, paraganglioma or pheochromocytoma. In some aspects, the cancer being treated is non-Hodgkin's lymphoma including but not limited to mycosis fungoides and Sezary syndrome. In some aspects, the cancer being treated is a soft tissue sarcoma. Examples include angiosarcoma, unresectable or progressive retroperitoneal/intra-abdominal soft tissue sarcoma, rhabdomyosarcoma, extremity/superficial trunk and/or head and neck cancer, or solitary fibrous tumor/hemangiopericytoma. In some aspects, the cancer being treated is bone cancer. Examples include Ewing's sarcoma and mesenchymal chondrosarcoma. In some aspects, the cancer being treated is uterine sarcoma, small cell lung cancer (SCLC) or Zollinger-Ellison syndrome. In some aspects, the cancer being treated in accordance with the invention is a gynecologic cancer (e.g., cancers of the female reproductive system) including, but not limited to ovarian cancer, cancer of the fallopian tube(s), peritoneal cancer and breast cancer. In some aspects, the cancer being treated in accordance with the invention is ovarian cancer. In additional aspects, the cancer is selected from small cell lung cancer (SCLC), Neuroendocrine Tumor, Ovarian Cancer, Sarcoma, Endometrial cancer, and Biliary tumors.

In some aspects, a cancer being treated in accordance with the invention is glioblastoma. Brain tumors spread extensively within the brain but do not usually metastasize outside the brain. Gliomas are very invasive inside the brain, even crossing hemispheres. They do divide in an uncontrolled manner, though. Depending on their location, they can be just as life threatening as malignant lesions. An example of this would be a benign tumor in the brain, which can grow and occupy space within the skull, leading to increased pressure on the brain.

Also provided are kits comprising the pharmaceutical compositions typically comprise a therapeutically effective amount of one or more agents used in the combination therapies of the invention described herein. Kits typically include a label indicating the intended use of the contents of the kits and instructions for use.

### EXAMPLES

The following examples are offered by way of illustration and are not to be construed as limiting the invention as claimed in any way.

### Example 1: Development of Manufacturing Strategies for iPSC-Based Gamma-Delta T-Cells

We have generated iPSCs and differentiated them in a stepwise manner into hematopoietic progenitor cells (HPCs), progenitor T-cells and cytotoxic T lymphocytes through a feeder-free multi-step differentiation process.

Methods: Precursor cells were obtained from healthy donors. γδ T cells were reprogrammed into iPSCs using non-integrating vectors encoding Oct3/4, Sox2, Kfl4, and c-Myc. Feeder-free multi-step differentiation strategies were established and applied to generate multiple effector cell populations. Cells were thereby differentiated from iPSC via hematopoietic progenitor cells (HPCs) to progenitor T-cells and further to gd T-cell subsets. Multiplex genomic PCR assays and Sanger sequencing were performed to examine rearrangements at TCRG and TCRD gene loci. Karyotype of iPSC lines were evaluated with the G-band karyotype analysis. Flow cytometry was used to identify cell surface markers for iPSCs, including pluripotency markers, (Tra-1-60, OCT3/4& SSEA4), HPC markers (CD34, CD43), Progenitor T-cell markers (CD5, CD7), and iPSC-derived γδ (i-γδ) T cell markers (CD3/γδ TCR/Vd1 TCR). To measure cytotoxicity, glioblastoma cells (U87-GFP) were co-cultured with i-γδ T cells at the indicated Effector: Target (E:T) ratios for 5 hours in vitro, stained with 7AAD and acquired by flow cytometry. GFP+/7AAD+ cell percentages indicate cytotoxicity. Statistical significance of changes observed between groups (three biological replicates per group) was determined by Student's t-test utilizing GraphPad Prism v9.

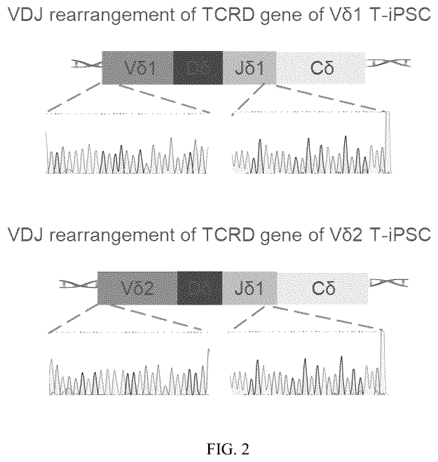

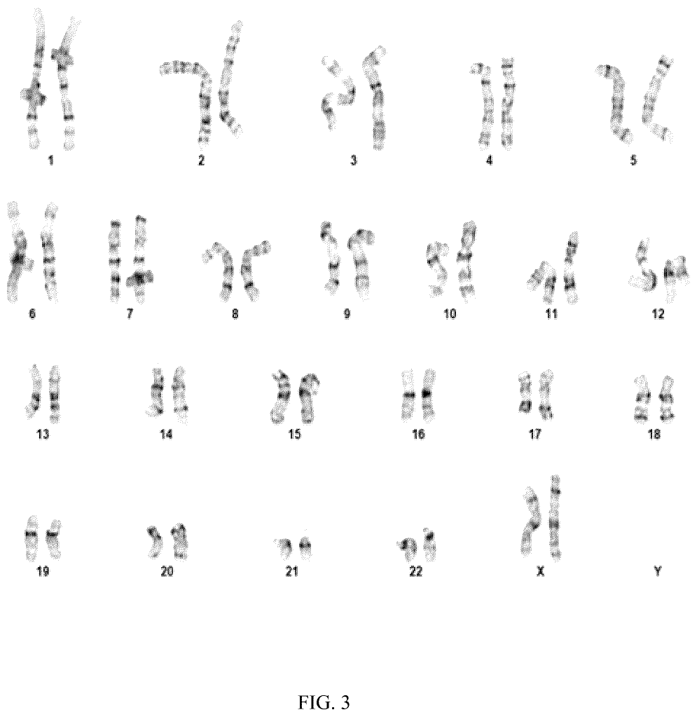

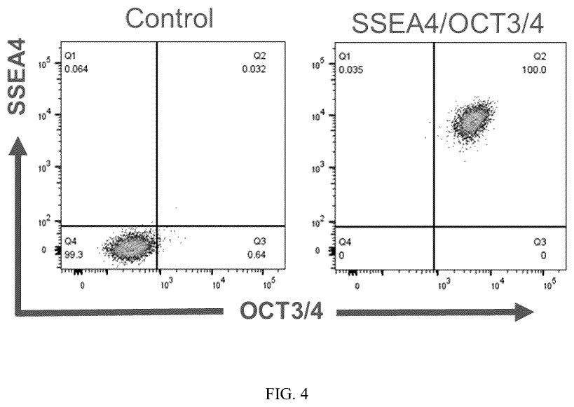

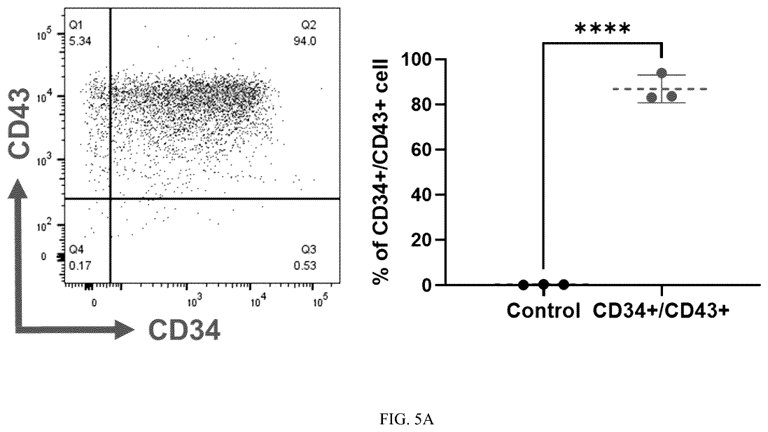

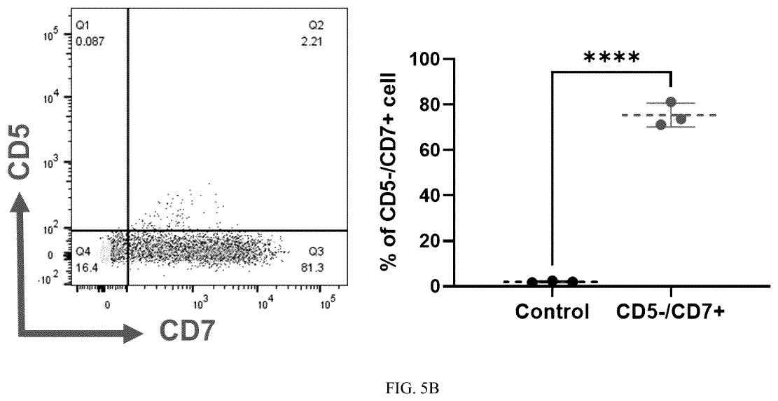

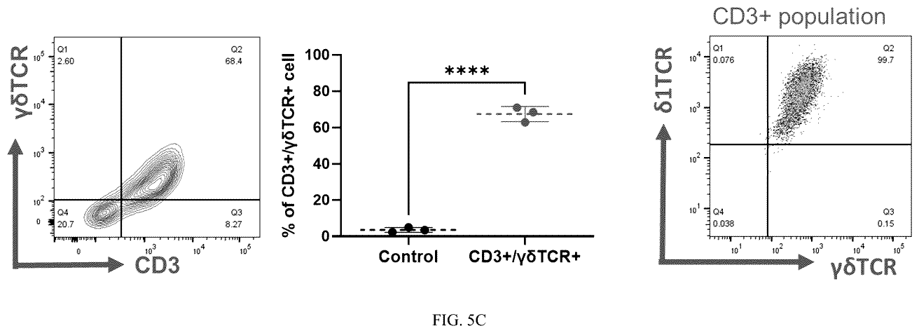

Results: Both γδ1 T-iPSC and γδ2 T-iPSC lines were generated which harbor the rearrangements of the TCRG and TCRD gene regions (FIG. 2). The γδ1 T-iPSC line was identified as Vγ2- to Jγ1/2 and Vδ1-to-Jδ1 recombination, and the γδ2 T-iPSC line was identified as γδ2 T-iPSC recombination. The γδ T-iPSCs lines were characterized as having normal karyotypes (FIG. 3) and highly expressing pluripotent markers (Tra-1-60, OCT3/4& SSEA4) (FIG. 4). 98% γδ1T-iPSCs are OCT3/4 & SSEA4 double positive. 99.7% γδ2T-iPSCs are OCT3/5 & SSEA4 double positive. The early passage γδ T-iPSC, Vδ1 T-iPSCs, were expanded and went through the multi-step differentiating process with a feeder-free culture system. At different stages of the process, high-yields of CD34+/CD43+ HPCs (86.9%), CD5−/CD7+ progenitor T cells (75.4%), and CD3+/γδ TCR+i-γδ T-cells (iPSC-derived γδ T cells) (50.9%) were obtained (FIGS. 5A-5C).

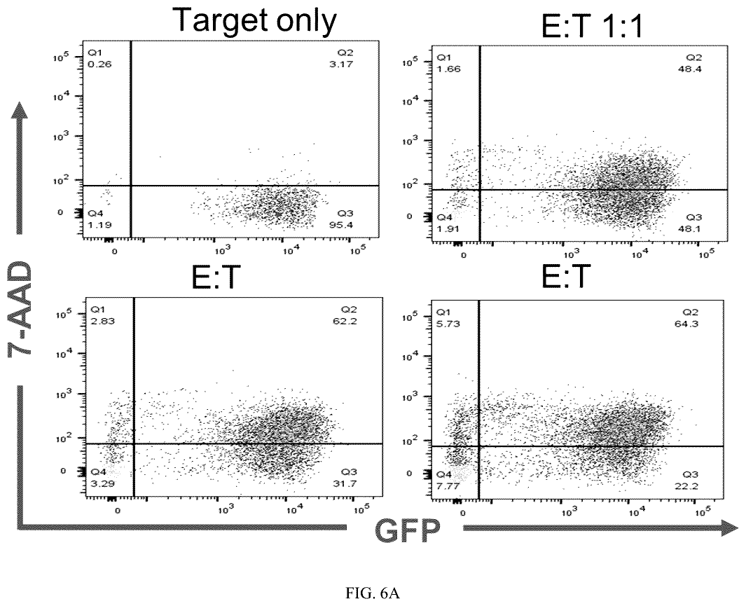

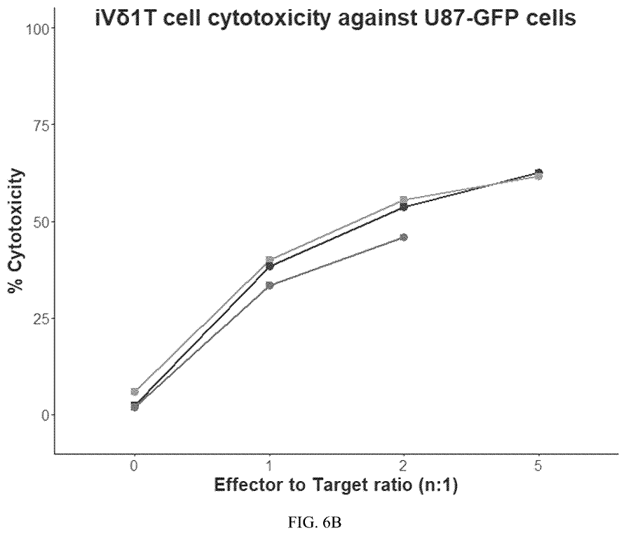

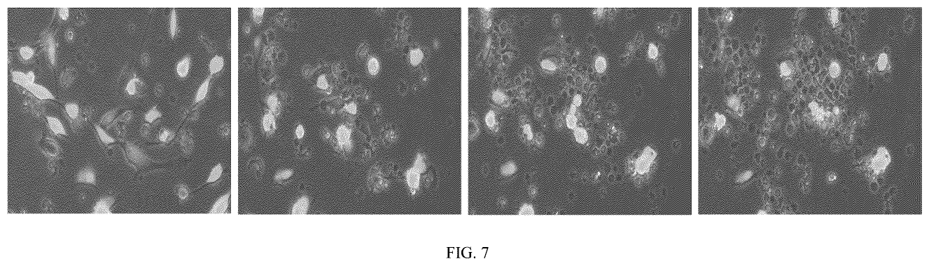

i-Vδ1 T cells are highly cytotoxic against U87-GFP cells in a dose-dependent manner. GFP+/7-AAD+ cell percentages indicate cytotoxicity (FIGS. 6A and 6B). U87-GFP cells were co-cultured with i-Vd1 T cells at E:T of 5:1. Images were taken minute (min) for a total of 300 min. Panel images taken at different time points show that U87-GFP cells were killed by i-Vδ1 T cells during co-culture (FIG. 7).

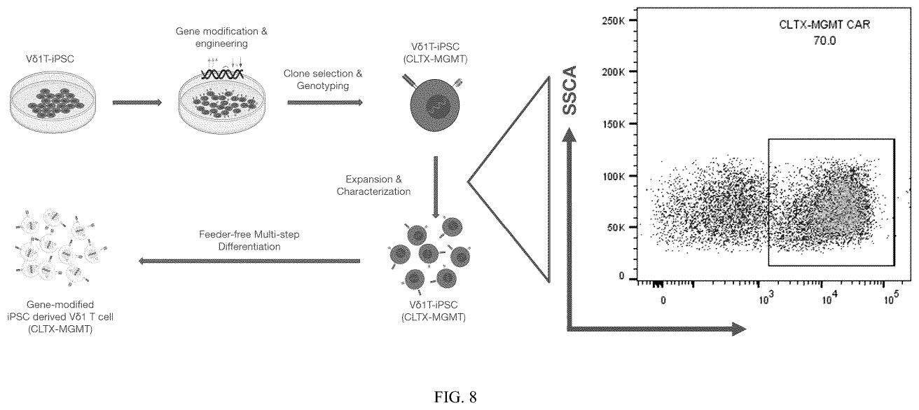

In addition, as shown in FIG. 8, Vδ1 T-iPSCs were transduced with 2×CLTX-noZ-MGMT CAR lentiviral vector (the vector encoding a CAR with two CLTX peptides and no CD3z signaling domain, and comprising MGMT, as described, for example, in WO2022159582; the contents of which are expressly incorporated by reference herein) with high efficiency (70%). These genome modified Vδ1 T-iPSCs will be cloned and characterized before going through the multi-step differentiation process to further enhance the targeting and cytotoxicity of the i-Vδ1T cells against glioblastoma and other solid tumors.

iPSC-derived γδ1 T-cells can provide an unlimited cell source to treat tumors while minimizing the risk for GvHD. An “off-the-shelf” platform was developed that enables manufacturing of different cell populations from parental iPSC lines including HPCs, progenitor T-cells, γδ1 and γδ2 T-cells. From healthy donor precursor cells, Vδ1-T and Vδ2-T iPSC lines with normal karyotypes that highly express pluripotency markers were generated. Through the feeder free multi-step differentiation process, γδT-iPSCs were differentiated into hematopoietic progenitor cells (HPCs), progenitor T cells and cytotoxic γδT lymphocytes in a stepwise manner. The iPSC-derived Vδ1 T lymphocytes showed high levels of dose-dependent cytotoxicity against U-87 glioblastoma cell lines. Genome modifications including the addition of chemotherapy resistance, CARs and other edits on i-γδ T cells may be necessary to successfully target solid tumor cancers. iPSC derived γδ T cells offer a promising future for scaling, manufacturing, enhancing targeting and killing efficacy, T cell fitness and overcoming the immunosuppressive tumor microenvironment (TME) for cellular immunotherapies against solid tumors. In summary, the multi-step feeder-free differentiation process produced both Vδ1+ and Vδ2+γδ T-cells and will pave the way toward novel genetic engineering and immunotherapies that can be applied towards various types of cancers.

While this invention has been particularly shown and described with references to preferred embodiments thereof, it will be understood by those skilled in the art that various changes in form and details may be made therein without departing from the scope of the invention encompassed by the appended claims. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference. The relevant teachings of all patents, published applications and references cited herein are incorporated by reference in their entirety.

1. A method of generating functional iPSC-derived γδ T-cells comprising the steps of:
a. preparing a population of precursor cell-derived induced pluripotent stem cells (iPSCs), the method comprising the steps of:
i. isolating a population of precursor cells and culturing the precursor cells in a culturing medium, wherein the precursor cells are human somatic cells;
ii. transducing the precursor cells with a non-integrating virus vector encoding a plurality of reprogramming factors, optionally comprising one or more of Oct3/4, Sox2, Kfl4 and c-Myc; and
iii. culturing the transduced precursor cells under conditions suitable for reprogramming the cells to pluripotency to obtain the population of precursor cell derived-iPSCs; and
b. differentiating the precursor cell-derived iPSCs to CD34+ hematopoietic progenitor cells (HPCs) and generating progenitor T-cells and functional iPSC-derived γδ T-cells therefrom under feeder-free conditions, wherein the iPSC-derived γδ T-cells comprise γδ1 T-cells and/or γδ2 T-cells.

2. The method of claim 1, wherein the non-integrating virus vector is Sendai virus.

3. The method of claim 1, wherein the precursor cells are selected from the group consisting of fibroblasts, adult stem cells, cord blood cells, Sertoli cells, granulosa cells, neurons, pancreatic islet cells, epidermal cells, epithelial cells, endothelial cells, hepatocytes, hair follicle cells, keratinocytes, hematopoietic cells, melanocytes, chondrocytes, lymphocytes, macrophages, monocytes, mononuclear cells, cardiac muscle cells, and skeletal muscle cells.

4. The method of claim 1, wherein the precursor cells are selected from the group consisting of cord blood cells, bone marrow cells, skin cells, and lymphocytes.

5. The method of claim 4, wherein the precursor cells are cord blood cells.

6. The method of claim 3, wherein the precursor cells are human keratinocytes.

7. The method of claim 4, wherein the precursor cells are lymphocytes.

8. The method of claim 7, wherein the lymphocytes are isolated from cord blood, peripheral blood, or bone marrow.

9. The method of claim 7, wherein the lymphocytes are γδ T-cells or NK cells.

10. The method of claim 9, wherein the lymphocytes are isolated from cord blood.

11. The method of claim 9, wherein the lymphocytes are isolated from bone marrow.

12. The method of claim 9, wherein the lymphocytes are isolated from peripheral blood.

13. The method of claim 1, wherein the reprogramming factors comprise one or more of Oct3/4, Sox2, Kfl4 and c-Myc.

14. The method of claim 13, wherein the reprogramming factors comprise Oct3/4, Sox2, Kfl4 and c-Myc.

15. The method of claim 1, further comprising engineering the precursor-cell derived iPSCs to express a polypeptide.

16. The method of claim 15, wherein the engineering or genetically modifying comprises genome editing.

17. The method of claim 15, wherein the precursor-cell derived iPSCs are engineered to express a polypeptide selected from the group consisting of a chimeric antigen receptor (CAR), a survival factor, and a combination thereof, wherein the survival factor is a polypeptide the confers resistance to a chemotherapeutic agent.

18. A method of preparing a population of precursor cell-derived induced pluripotent stem cells (iPSCs), comprising:
i. isolating a population of precursor cells and culturing the precursor cells in a culturing medium, wherein the precursor cells are human somatic cells;
ii. transducing the precursor cells with a non-integrating virus vector encoding a plurality of reprogramming factors, optionally comprising one or more of Oct3/4, Sox2, Kfl4 and c-Myc; and
iii. culturing the transduced precursor cells under conditions suitable for reprogramming the cells to pluripotency to obtain the population of precursor cell derived-iPSCs.

19. A cell population comprising precursor cell-derived iPSCs prepared by the method of claim 18.

20. A cell population comprising functional iPSC-derived γδ T-cells prepared by the method of claim 1, wherein the functional iPSC-derived γδ T-cells are differentiated from the iPSCs and wherein the γδ T-cells comprise γδ1T-cells and/or γδ2 T-cells.

21. The cell population of claim 20, wherein the γδ T-cells express a polypeptide selected from the group consisting of a chimeric antigen receptor (CAR), a survival factor, and a combination thereof.

22. The cell population of claim 21, wherein the CAR is directed to a tumor antigen.

23. The cell population of claim 21, wherein the CAR comprises an extracellular antigen-binding domain comprising a CLTX peptide.

24. The cell population of claim 23, wherein the extracellular antigen-binding domain comprises at least two CLTX peptides.

25. The cell population of claim 20, wherein the γδ T-cells express a survival factor, wherein the survival factor is a polypeptide that confers resistance to a chemotherapeutic agent.

26. A method of treating cancer in a patient in need thereof comprising administering to said patient an effective amount of a composition comprising the cell population of claim 20.

27. The method of claim 26, wherein the cancer is a solid tumor.

28. The method of claim 20, the method further comprising co-administering further comprising co-administering to said subject the chemotherapeutic agent in an amount sufficient to increase stress antigen expression on the cancer or tumor cells and wherein the cells express a polypeptide that confers resistance to the chemotherapeutic agent.
